(19) United States
(12) Patent Application Publication
(10) Pub. No.: US 2015/0057517 A1
(43) Pub. Date: Feb. 26, 2015

(54) SAMPLE CAPTURE DEVICE AND SYSTEMS AND METHODS OF USING SAME

(71) Applicant: UNIVERSITY OF UTAH RESEARCH FOUNDATION, Salt Lake City, UT (US)

(72) Inventors: Leonard F. Pease, Bountiful, UT (US); Kathryn Peterson, Salt Lake City, UT (US); Hedieh Saffari, Salt Lake City, UT (US); Joseph R. Heath, Salt Lake City, UT (US); Gerald Gleich, Salt Lake City, UT (US); Kristin Leiferman, Salt Lake City, UT (US)

(21) Appl. No.: 14/388,406

(22) PCT Filed: Mar. 27, 2013
(86) PCT No.: PCT/US13/34170

§ 371 Date: Sep. 26, 2014

Related U.S. Application Data
(60) Provisional application No. 61/685,917, filed on Mar. 27, 2012.
(60) Provisional application No. 61/744,167, filed on Sep. 19, 2012.

(51) Int. Cl. A61B 10/04; A61B 5/145; A61B 1/273; A61B 1/005; A61B 1/008; A61B 5/00; A61F 13/15; A61B 1/05

(52) U.S. Cl. CPC A61B 10/04; A61F 13/15; A61B 5/14539; A61B 1/05; A61B 1/0055; A61B 1/0057; A61B 1/008; A61B 5/4233; A61B 1/2733; A61B 2010/0216
USPC 600/350; 600/562; 600/572; 600/569

(57) ABSTRACT

Disclosed are devices, compositions, and methods for diagnosing eosinophilic esophagitis in a subject. Also disclosed are methods for monitoring the course of eosinophilic esophagitis in a subject before, during, and after treatment.

### RELATED APPLICATIONS

This application claims the benefit of U.S. Provisional Patent Application No. 61/685,917, filed on Mar. 27, 2012, and U.S. Provisional Patent Application No. 61/744,167, filed on Sep. 19, 2012. U.S. Provisional Patent Application No. 61/685,917 and U.S. Provisional Patent Application No. 61/744,167 are hereby incorporated herein by reference in their entirety.

### BACKGROUND

Eosinophilic esophagitis (EoE) is a chronic disease of the esophagus that affects over 300,000 patients in the U.S. alone. Symptoms include dysphagia (difficulty swallowing liquids or solids or both, >90%), food impaction (solid food sticks in the esophagus, 50%), odynophagia (painful swallowing), heartburn (33%), chest pain, asthma (50%), diarrhea, and vomiting (Gonsalves, Kahrilas, Am J Gastroenterol, 2009). The disease primarily occurs in males (75%) with a mean age between 36 and 42 years in westernized countries. While present in adults, the disease can also manifest in children. The symptoms of EoE are similar to an atopic allergenic inflammatory condition of the esophagus, affecting up to 10% of adults presenting for upper endoscopy (Mackenzie, Aliment Ther Pharmacol, Gastroenterol, 2008).

Although the source or sources of this disease have not been conclusively identified, investigators have identified several contributing factors. Genetic predisposition may be at work in this disease, at least in part, due to the increased incidence in first degree relatives of EoE patients relative to the general population. Environmental causes may also be important as allergens (i.e., food and aero-allergens) contribute in up to 97% of cases in children (Liacouras, Clin Gastro Hep, 2005). Fogg et al. (2003) observed worsening of EoE during the pollination season in an allergic patient. Wang et al. (2007) subsequently identified a seasonal variation in identification and severity of the disease in children. Furthermore, Mishra et al. (2001) determined that intranasal administration of Aspergillus fumigatus in a mouse model replicated the esophageal eosinophilic infiltrate seen in EoE. However, EoE is not simply a seasonal allergy of the esophagus.

Food allergies also play an important role in both adult and pediatric EoE. Markowitz et al. (2003) found resolution of esophageal eosinophilia after 4 weeks of amino acid-based elemental diet in 49/51 pediatric patients. In the largest analysis to date, Liacouras et al. (2005) found a 97% response to an elemental diet in a cohort of 160 children with EoE. However, preliminary data on an elimination diet in adults found less robust responses than those observed in children. The six-food elimination diet (Gonsalves et al., 2012) demonstrated improvement in 78% and 33% complete resolution rate. Elemental diet in adults results in substantial improvement in eosinophilia after 4 weeks in 72% of patients (Peterson, 2013). Responses to skin prick testing in adults undergoing food elimination diets suggest a multi-modal (IgE and non-IgE mediated) immunological process, and murine models find both aero-allergens and food each play significant roles (Mishra, J Clin Invest, 2001).

In all cases, detection of EoE via a form of endoscopy known as esophagoduodenoscopy (EGD) remains essential. In this procedure, a small tube with a camera on the distal end is passed into the esophagus, stomach, and first portion of the small intestine to visualize the mucosal surfaces of these organs. In EoE, the inflammation occurs in various parts of the esophagus; there is approximately equal incidence in the proximal, distal, or both portions of the esophagus being affected (Gangotena, Am J Gastroenterol, 2007) within cohorts, but such infiltrate varies in each individual with many demonstrating a less intense infiltrate proximally. EoE also affects the luminal caliber and phenotypic appearance of the esophagus. Pronounced rings or furrows can develop into strictures that close off the esophagus, resulting in odynophagia, dysphagia, food impaction, and emergency hospital visits. The areas of inflammation are not evenly distributed throughout an affected esophagus, as the disease often presents in patches or select segments of the 25-30 cm long adult esophagus.

Although EGD is a key tool in the identification of EoE, some cases (15-20%) may never present as a “ringed-esophagus” during EGD. Mackenzie et al. (2008). A conclusive means currently available to clinicians to positively identify EoE is to detect the presence of eosinophils in biopsy specimens. Tissue samples may be collected during EGD and then examined with traditional histological analysis to confirm or reject a case of EoE. However, the patchy nature of the disease complicates collection of tissue samples for biopsy. When clinical suspicion for EoE is high, consensus practice requires sampling at 4 to 5 sites throughout the esophagus. However, five 2 mm biopsy specimens represent less than 0.7% of the 20- to 25-cm-long adult esophageal mucosa and might result in underdiagnosis of EoE if mucosal eosinophilia is particularly patchy. Specific disease phenotypes (.e., rings, lines, furrows, white spots, or plaques) aid physicians in determining where and how many biopsies to perform based on EGD-observed phenotypes, which are strong indicators of eosinophil density. For example, biopsies to collect tissue samples are often collected from unaffected areas. For this reason, at least 4 (child) or 5 (adult) biopsy specimens are required to confirm each case of EoE (Gonsalves Gastrointestinal Endosc, 2006; Shah Am J Gastroenterol, 2009). Furthermore, additional biopsies are required to evaluate the effectiveness of each treatment proposed. This repeated need for endoscopic removal of tissue poses a financial hardship for the patient, and the procedure can be painful, requiring sedation and/or anesthesia.

The key element for diagnosing EoE in a biopsy specimen is the presence of eosinophils. Normal esophageal tissue does not contain eosinophils (Kato et al., 1998). These white blood cells were named for their affinity for the red dye eosin. Normally, eosinophils reside in the blood stream, stomach, small and large intestine, and lymphatic system (Kato et al., 1998) but infiltrate pathologically into the esophagus in EoE. In biopsy samples, an eosinophil can be identified as a cell 12-17 μm in diameter with a bi-lobed nucleus and cytoplasmic granules staining red with acidic dyes, for example eosin. A tissue count of eosinophils in excess of 15 per field of view at high microscope power (greater than 15 per high-powered field (hpf)) indicates EoE. Some clinical evidence suggests that inflammation increases with eosinophil concentration.

A distinctive characteristic of eosinophils is their granules, which comprise markedly cationic proteins, each of which is composed of a core and a matrix. The core consists primarily of major basic protein 1 (MBP-1); the matrix consists of eosinophil peroxidase (EPO) and eosinophil derived neurotoxin (EDN) (Peters et al., 1986), inter alia. MBP-1 is a highly basic (isoelectric point approaching 12) 13.8 kDa protein with 5 unpaired cysteins that accounts for about 55% of the granule's protein (Gleich et al., 1974; Gleich et al., 1976). It is a member of the C-type lectin family (lectins bind sugars) and has the highest concentration in the eosinophil granule on a per molecule basis (Abu-Ghazaleh et al., 1992). EPO has the highest concentration in the granule on a per mass basis, while EDN and ECP are members of the RNAse 2 family (Gleich et al., 1986). Upon degranulation, an eosinophil releases each of these proteins into the surrounding tissues. Of these, only MBP-1 stimulates histamine release (O'Donnell et al., 1983). MBP-1 also exfoliates bronchial epithelial cells (Frigas et al., 1980) and causes bronchial hyper-reactivity (Gundel et al., 1991), whereas both MBP-1 and EPO provoke transient bronchial constriction (Gundel et al., 1991). These proteins are found in abundance in biopsies in eosinophilic esophagitis (Kephart, Am J Gastroenterol, 2010).

Currently, as symptoms are unable to predict the severity of eosinophilic involvement, the only way to adequately monitor the extent and severity of the disease is through invasive upper endoscopy with biopsy. Often, in food re-introduction and therapeutic evaluation, this results in several upper endoscopies per year for patients. Due to the cost, invasiveness, and discomfort experienced via this method of monitoring, patients become non-compliant, and subsequently the disease is not adequately tracked. Additionally, there is a lack of sensitivity of biopsies in detecting and understanding such a patchy disease because biopsies histologically characterize only <0.03% of the entire esophagus.

Despite the rapidly growing incidence of EoE, state-of-the-art diagnostic techniques remain inadequate to fully characterize this disease. As such, there exists a need to develop a precise and comprehensive technique to image and map the distribution of inflammation and deposition of eosinophil granule proteins. Such techniques will provide a tool to diagnose EoE, track disease activity in response to various treatment regimens, and obtain previously unreachable insight into the development and progression of EoE pathophysiology.

### SUMMARY

In accordance with the purposes of this invention, as embodied and broadly described herein, disclosed, in one aspect, is a mucosal tissue sample capture device for insertion within the esophagus of a subject. The sample capture device can have a central axis. The sample capture device can include an elongate conduit that surrounds the central axis. The elongate conduit can define a central bore and have an outer surface, a first end, and an opposed, insertional end. The sample capture device can further include a central wire configured for selective movement relative to the central axis. At least a portion of the central wire can be positioned within the central bore of the elongate conduit. The sample capture device can further include at least one sheath operatively coupled to the outer surface of the elongate conduit. The sample capture device can still further include at least one capture assembly operatively coupled to the central wire. Selective axial movement of the central wire can effect axial and radial movement of each capture assembly. Each capture assembly can be axially moveable about and between an enclosed position and an open position. Each capture assembly can also be radially moveable about and between a retracted position and a deployed position.

Each capture assembly can include a plurality of buckling elements that are azimuthally spaced from each other relative to the central axis. Each buckling element can have a first end and an opposed second end. Each capture assembly can further include a first crimping element and a second crimping element. The first crimping element can be operatively coupled to the first ends of the plurality of buckling elements and to the central wire. The second crimping element can be operatively coupled to the second ends of the plurality of buckling elements such that the central wire is axially moveable relative to the second crimping element. In the enclosed position, each capture assembly of the at least one capture assembly can be positioned within a respective sheath of the at least one sheath. In the open position, each capture assembly of the at least one capture assembly can be axially advanced beyond a respective sheath such that the capture assembly is positioned outside the sheath. In the deployed position, the first and second crimping elements can compress the plurality of buckling elements of each capture assembly such that the plurality of buckling elements extend outwardly relative to the central axis. Optionally, each buckling element can include at least one capture element that is configured to capture a mucosal tissue sample of the subject.

In another aspect, disclosed is a mucosal tissue sample capture device for insertion within the esophagus of a subject. The sample capture device can have a central axis. The sample capture device can include first and second central wires configured for selective movement relative to the central axis. The sample capture device can also include at least one sheath operatively coupled to the first central wire. Selective movement of the first central wire can effect movement of each sheath of the at least one sheath about and between a closed position and an open position. Additionally, the sample capture device can include at least one capture assembly operatively coupled to the second central wire. Selective movement of the second central wire can effect movement of each capture assembly of the at least one capture assembly about and between a retracted position and a deployed position.

Each capture assembly can include a plurality of buckling elements that are azimuthally spaced from each other relative to the central axis. Each buckling element can have a first end and an opposed second end. Each capture assembly can further include a first crimping element and a second crimping element. The first crimping element can be operatively coupled to the first ends of the plurality of buckling elements such that the first and second central wires are axially moveable relative to the first crimping element. The second crimping element can be operatively coupled to the second ends of the plurality of buckling elements and the second central wire such that the first central wire is axially moveable relative to the second crimping element. In the closed position of the at least one sheath, each capture assembly can be positioned within a respective sheath. In the open position of the at least one sheath, each sheath can be axially advanced relative to a respective capture assembly such that the capture assembly is positioned outside the sheath. In the deployed position, the second crimping element of each capture assembly compresses the plurality of buckling elements of the capture assembly such that the plurality of buckling elements extend outwardly relative to the central axis. Optionally, each buckling element can include at least one capture element that is configured to capture a mucosal tissue sample of the subject.

Also disclosed, in an additional aspect, is a method of diagnosing eosinophilic esophagitis in a subject, comprising detecting an eosinophil granule protein in the mucosal tissue of the esophagus in a subject, comprising: a) obtaining a mucosal tissue sample from the esophagus in the subject using the disclosed sample capture device; b) contacting the mucosal tissue sample with a detectable composition ex vivo under conditions wherein the detectable composition can bind to an eosinophil granule protein to form a detectable composition/eosinophil granule protein complex; and c) detecting the detectable composition/eosinophil granule protein complex in the mucosal tissue sample of the esophagus, whereby detecting the detectable composition/eosinophil granule protein complex in the mucosal tissue sample of the esophagus diagnoses eosinophilic esophagitis in the subject.

Further disclosed in an additional aspect is a method of diagnosing eosinophilic esophagitis in a subject, comprising detecting an eosinophil granule protein in the mucosal tissue of the esophagus in a subject, comprising: a) obtaining a mucosal tissue sample from the esophagus in the subject using the disclosed sample capture device; b) contacting the mucosal tissue sample with a detectable composition ex vivo under conditions wherein the detectable composition can physically, chemically, or physicochemically interact with an eosinophil granule protein to form a detectable composition/eosinophil granule protein complex; and c) detecting the detectable composition/eosinophil granule protein complex in the mucosal tissue sample of the esophagus, whereby detecting the detectable composition/eosinophil granule protein complex in the mucosal tissue sample of the esophagus diagnoses eosinophilic esophagitis in the subject.

### BRIEF DESCRIPTION OF THE DRAWINGS

The accompanying figures, which are incorporated in and constitute a part of this specification, illustrate several aspects and together with the description serve to explain the principles of the invention.

FIG. 1 depicts exemplary configurations of a sample capture device having a single central wire and a single capture assembly as disclosed herein. FIG. 1A is a partial cross-sectional view of an exemplary sample capture device with distance markings as disclosed herein. FIG. 1B is a partial cross-sectional view of an exemplary sample capture device having a pH probe, a microchip, and an indicator light as disclosed herein.

FIG. 2 is a partial cross-sectional view of an exemplary sample capture device having a single central wire, a plurality of capture assemblies, and a plurality of sheaths as disclosed herein.

FIG. 3 depicts an insertional portion of an exemplary sample capture device with a capture assembly positioned in enclosed, open, and deployed positions. FIG. 3A is a partial cross-sectional view of the capture assembly in the enclosed position. FIG. 3B is a partial cross-sectional view of the capture assembly in the open position. FIG. 3C is a partial cross-sectional view of the capture assembly in the deployed position.

FIG. 4 depicts various configurations of the insertional end of the sample capture device of FIG. 3. FIG. 4A is a partial cross-sectional view of an exemplary sample capture device having a terminal bead as disclosed herein. FIGS. 4B and 4C are partial cross-sectional views of exemplary sample capture devices having a terminal camera as disclosed herein. FIG. 4D is a partial cross-sectional view of an exemplary sample capture device having a terminal pH probe as disclosed herein. FIG. 4E is a partial cross-sectional view of an exemplary sample capture device having a conductivity probe as disclosed herein.

FIG. 5 depicts an insertional portion of an exemplary sample capture device with a capture assembly positioned in enclosed, open, and deployed positions. FIG. 5A is a partial cross-sectional view of the capture assembly in the enclosed position. FIG. 5B is a partial cross-sectional view of the capture assembly in the open position. FIG. 5C is a partial cross-sectional view of the capture assembly in the deployed position.

FIG. 6 depicts various configurations of the insertional end of the sample capture device of FIG. 5. FIG. 6A is a partial cross-sectional view of an exemplary sample capture device having a terminal bead as disclosed herein. FIGS. 6B and 6C are partial cross-sectional views of exemplary sample capture devices having a terminal camera as disclosed herein. FIG. 6D is a partial cross-sectional view of an exemplary sample capture device having a terminal pH probe as disclosed herein. FIG. 6E is a partial cross-sectional view of an exemplary sample capture device having a terminal pH probe secured to a secondary wire as disclosed herein. FIG. 6F is a partial cross-section view of an exemplary sample capture device having a conductivity probe as disclosed herein.

FIG. 7 depicts an exemplary actuator assembly in positions corresponding to the enclosed, open, and deployed positions of the capture assembly as disclosed herein. FIG. 7A is a partial cross-sectional view of the actuator assembly when the capture assembly is in the enclosed position. FIG. 7B is a partial cross-sectional view of the actuator assembly when the capture assembly is in the open position. FIG. 7C is a partial cross-sectional view of the actuator assembly when the capture assembly is in the deployed position.

FIG. 8 depicts an alternative configuration of an exemplary actuator assembly in positions corresponding to the enclosed, open, and deployed positions of the capture assembly as disclosed herein. FIG. 8A is a partial cross-sectional view of the actuator assembly when the capture assembly is in the enclosed position. FIG. 8B is a partial cross-sectional view of the actuator assembly when the capture assembly is in the open position. FIG. 8C is a partial cross-sectional view of the actuator assembly when the capture assembly is in the deployed position.

FIG. 9 depicts an insertional portion of an exemplary sample capture device with a capture assembly positioned in enclosed, open, and deployed positions. As shown, the sample capture device comprises first and second central wires. FIG. 9A is a partial cross-sectional view of the capture assembly in the enclosed position. FIG. 9B is a partial cross-sectional view of the capture assembly in the open position. FIG. 9C is a partial cross-sectional view of the capture assembly in the deployed position.

FIG. 10 depicts an exemplary actuator assembly in positions corresponding to the enclosed, open, and deployed positions of the capture assembly of FIG. 9 as disclosed herein. FIG. 10A is a partial cross-sectional view of the actuator assembly when the capture assembly is in the enclosed position. FIG. 10B is a partial cross-sectional view of the actuator assembly when the capture assembly is in the open position. FIG. 10C is a partial cross-sectional view of the actuator assembly when the capture assembly is in the deployed position.

FIG. 11 depicts an exemplary buckling element having a contact element and an opposed shim element. FIG. 11A depicts the buckling element in the retracted position, while FIG. 11B depicts the buckling element in the deployed position.

FIG. 12 depicts an exemplary buckling element having a relatively stiff contact element. FIG. 12A depicts the buckling element in the retracted position, while FIG. 12B depicts the buckling element in the deployed position.

FIG. 13 depicts an exemplary buckling element having a relatively limp contact element. FIG. 13A depicts the buckling element in the retracted position, while FIG. 13B depicts the buckling element in the deployed position.

FIG. 14 depicts an exemplary buckling element having a contact element, an opposed shim element, and an enclosing element as disclosed herein. FIG. 14A depicts the buckling element in the retracted position, while FIG. 14B depicts the buckling element in the deployed position.

FIG. 15 depicts an exemplary buckling element having a relatively stiff contact element and an enclosing element. FIG. 15A depicts the buckling element in the retracted position, while FIG. 15B depicts the buckling element in the deployed position.

FIG. 16 depicts an exemplary buckling element having a relatively limp contact element and an enclosing element. FIG. 16A depicts the buckling element in the retracted position, while FIG. 16B depicts the buckling element in the deployed position.

FIG. 17 depicts top perspective views of the buckling element of FIGS. 15 and 16. FIG. 17A depicts the buckling element in the retracted position, while FIG. 17B depicts the buckling element in the deployed position.

FIG. 18 depicts an exemplary capture assembly having a plurality of buckling elements in a staggered configuration as disclosed herein.

FIG. 19 depicts exemplary capture assemblies having contact elements with greater longitudinal length than the buckling elements of the capture assemblies. FIG. 19A depicts the capture assemblies in a retracted position. FIGS. 19B and 19C depict exemplary alternative configurations of the capture assemblies in the deployed position.

FIG. 20 depicts alternative configurations of the sample capture device in which a plurality of capture elements are secured directly to a central wire. FIG. 20A depicts a configuration of the sample capture device in which the capture elements are secured to the central wire with tension. FIG. 20B depicts a configuration of the sample capture device in which the central wire has a plurality of joints positioned between the capture elements as disclosed herein. FIGS. 20C and 20D are detailed mechanical views of the capture and buckling elements of FIGS. 20A and 20B. FIGS. 20E and 20F are isolated views of exemplary joints of the sample capture device of FIGS. 20A and 20B.

FIG. 21 depicts an exemplary capture assembly having pocketed crimping elements as disclosed herein. FIG. 21A depicts the capture assembly in a retracted position, while FIG. 21B depicts the capture assembly in a deployed position.

FIGS. 22A and 22B depict exemplary maps of esophageal disease that can be produced using a sample capture device having three capture assemblies, with each capture assembly having three buckling elements. FIG. 22A depicts a first map, while FIG. 22B depicts a second map taken at a later time.

FIGS. 23A and 23B depict exemplary maps of esophageal disease that can be produced using a sample capture device having three capture assemblies, with each capture assembly having four buckling elements. FIG. 23A depicts a first map, while FIG. 23B depicts a second map taken at a later time.

FIG. 24 shows the absorbance measurements for various capture elements that were acquired using the EPO detection system.

FIG. 25 shows the intensity change in an EPO assay. Detection solution absorbance measurements of 1:1000 dilution of EPO stock solution in 1× PBS are shown over time. The solution gets darker over time but changes color in the first couple of minutes, with and without the termination step.

FIGS. 26A and 26B exemplary outer sheaths extending along a portion of the length of the sample capture devices as disclosed herein. FIG. 26A depicts an exemplary outer sheath for the sample capture device of FIG. 1. FIG. 26B depicts an exemplary outer sheath for the sample capture device of FIG. 2.

FIG. 27 depicts an exemplary outer sheath extending along substantially the entire length of the sample capture devices as disclosed herein. FIG. 27A depicts an exemplary outer sheath for the sample capture device of FIG. 1. FIG. 27B depicts an exemplary outer sheath for the sample capture device of FIG. 2. As shown in FIG. 27B, the outer sheath can define portals to permit communication between the capture assemblies and the esophagus of a subject.

### DETAILED DESCRIPTION

What are needed in the art are devices, compositions, and methods for quickly diagnosing eosinophil degranulation-associated esophagitis in a subject and for monitoring the effectiveness of treatment in the subject in order to decrease suffering and cost and to increase subject compliance. Eosinophil degranulation-associated esophagitis is eosinophilic esophagitis (EoE). The disclosed devices, compositions, and methods can diagnose eosinophilic esophagitis in a subject within a few minutes, by detecting in an esophageal tissue sample ex vivo the presence of eosinophil granule proteins.

Thus, disclosed is the surprising discovery that detectable compositions can be used ex vivo to bind to and identify eosinophil granule proteins, which are absent in the normal esophagus but are deposited in and are associated with inflammation in the mucosal tissue of the esophagus after eosinophil degranulation in a subject with EoE.

The present invention may be understood more readily by reference to the following detailed description of various aspects of the invention and the Examples included therein and to the Figures and their previous and following description.

Before the present compounds, compositions, articles, devices, and/or methods are disclosed and described, it is to be understood that this invention is not limited to specific synthetic methods or specific detectable compositions, as such may, of course, vary. It is also to be understood that the terminology used herein is for the purpose of describing particular aspects only and is not intended to be limiting.

As used in the specification and the appended claims, the singular forms “a,” “an,” and “the” include plural referents unless the context clearly dictates otherwise. Thus, for example, reference to “a detectable composition” or a “detectable composition/eosinophil granule protein complex” can include mixtures of detectable compositions or mixtures of detectable composition/eosinophil granule protein complexes, respectively, and the like.

Ranges may be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant, both in relation to the other endpoint and independently of the other endpoint.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a detectable composition is disclosed and discussed and a number of modifications that can be made to a number of molecules including the detectable composition are discussed, each and every combination and permutation of the detectable composition and the modifications that are possible are specifically contemplated unless specifically indicated to the contrary. Thus, if a class of molecules A, B, and C is disclosed as well as a class of molecules D, E, and F and an example of a combination molecule A-D is disclosed, then even if each is not individually recited, each is individually and collectively contemplated. Thus, in this example, each of the combinations A-E, A-F, B-D, B-E, B-F, C-D, C-E, and C-F is specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application including, but not limited to, steps in the methods of making and using the disclosed detectable compositions and detectable composition/eosinophil granule protein complexes. Thus, if there are a variety of additional steps that can be performed, it is understood that each of these additional steps can be performed with any specific aspect or combination of aspects of the disclosed methods and that each such combination is specifically contemplated and should be considered disclosed.

Those skilled in the art will recognize, or be able to ascertain using no more than routine experimentation, many equivalents to the specific aspects of the methods and compositions described herein. Such equivalents are intended to be encompassed by the appended claims.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the disclosed methods and compositions, the particularly useful methods, devices, and materials are as described.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents.

It is understood that the disclosed devices, methods, and compositions are not limited to the particular methodology, protocols, and reagents described, as these may vary. It is also to be understood that the terminology used herein is for the purpose of describing particular aspects only and is not intended to limit the scope of the present invention which will be limited only by the appended claims.

In this specification and in the claims which follow, reference will be made to a number of terms which shall be defined to have the following meanings. The word “comprise” and variations of the word, such as “comprising” and “comprises,” means “including, but not limited to” and is not intended to exclude, for example, other additives, components, integers or steps. “Optional” or “optionally” means that the subsequently described event or circumstance may or may not occur and that the description includes instances where said event or circumstance occurs and instances where it does not. As used herein, by “subject” is meant an individual. A subject can be a mammal such as a primate, for example, a human. The term “subject” includes domesticated animals such as cats, dogs, etc., livestock (e.g., cattle, horses, pigs, sheep, goats, etc.), and laboratory animals (e.g., mice, rabbits, rats, gerbils, guinea pigs, possums, etc.). As used herein, the terms “subject” and “patient” are interchangeable.

As used herein, the term “insertional” refers to the end of a first element that is configured for insertion into a second element. For example, as used herein, an “insertional” end of an elongate conduit can be configured for insertion into the lumen defined by the esophagus of a subject.

As used herein, the term “azimuthally” refers to an imaginary arc that is (a) defined within a plane transverse to the central axis of the capture devices disclosed herein and (b) symmetrically spaced from the central axis, with the central axis serving as the center point of the arc. In exemplary aspects, the imaginary arc can be a circle or an ellipse. Thus, as used herein, items are azimuthally spaced from one another when the items are positioned at distinct locations along the imaginary arc. It is contemplated that the relative angular orientation of the azimuthally spaced items can be measured with respect to the central axis of a capture device as disclosed herein.

Disclosed are devices, compositions, and methods for diagnosing EoE in a subject and for monitoring the course of the disease before, during, and after treatment of the disease. As further described herein, the disclosed devices, systems, and methods can be used to detect an eosinophil granule protein in the mucosal tissue of the esophagus in a subject.

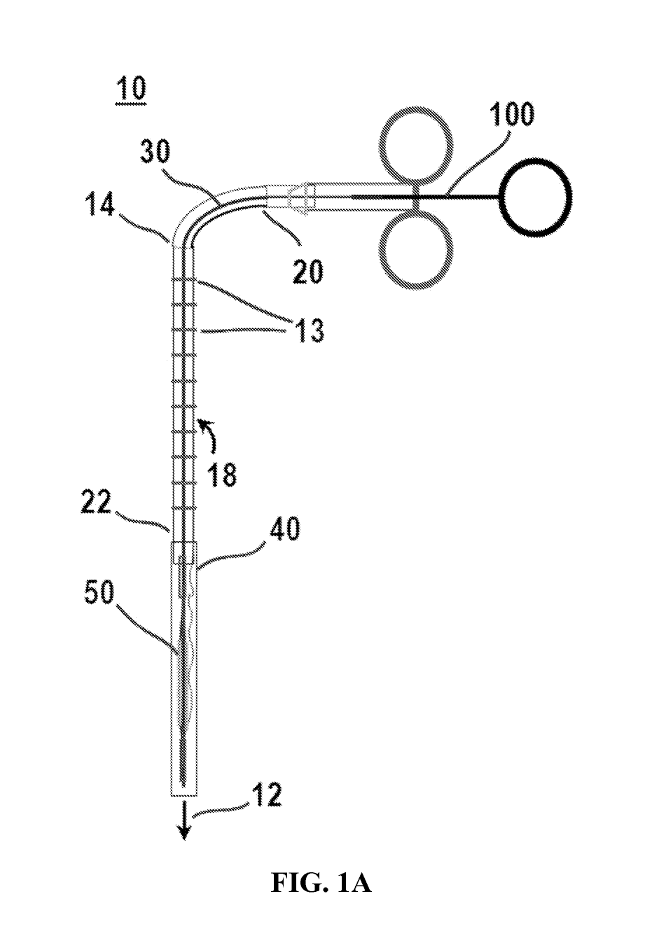

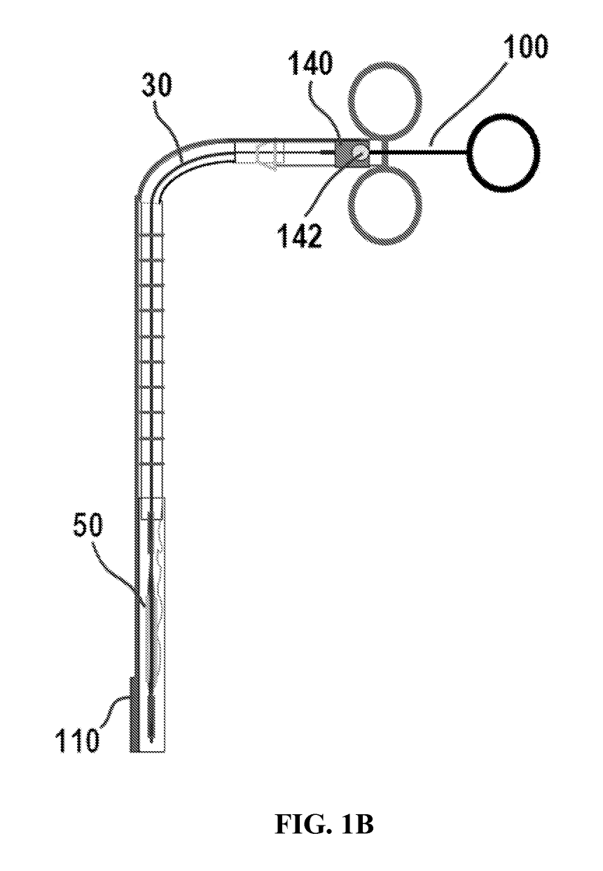

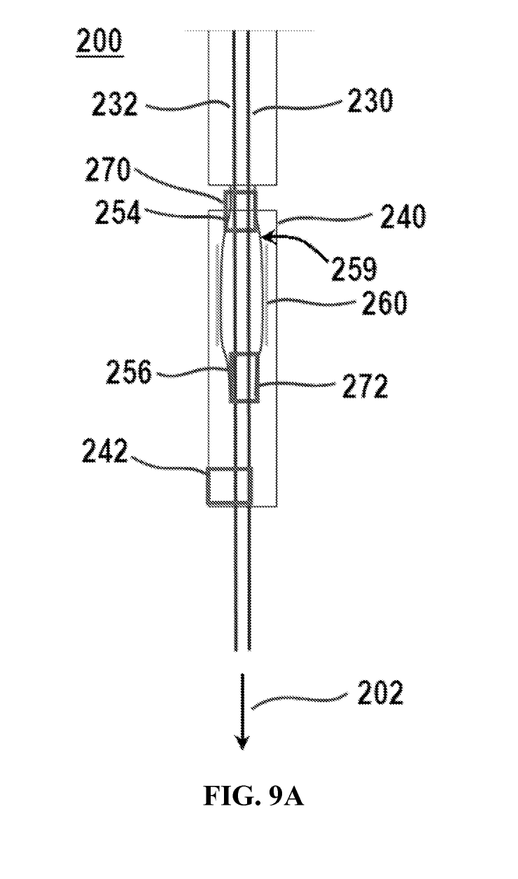

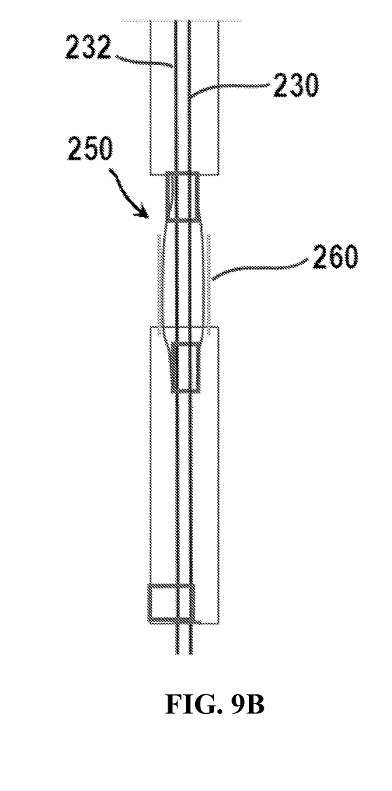

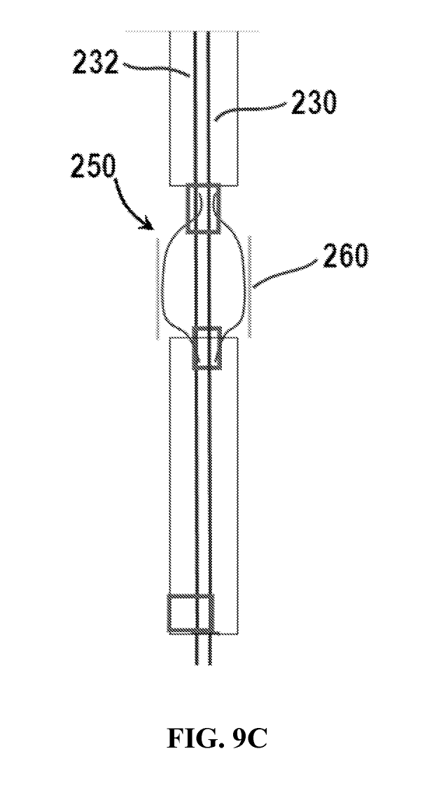

In exemplary aspects, a mucosal tissue sample capture device 10, 200 can be used to capture a mucosal tissue sample from the esophagus of the subject. In these aspects, the sample capture device 10, 200 can be configured for insertion within the lumen of the esophagus of the subject. It is contemplated that the sample capture device 10, 200 can comprise biocompatible materials that are capable of conforming to the shape of the esophagus such that the sample capture device can be selectively advanced within the esophagus of the subject as described herein. As shown in FIGS. 1A and 9A, the sample capture device 10, 200 can have a central axis 12, 202. As further described herein, the sample capture device 10, 200 can comprise at least one capture assembly 50, 250 that is moveable about and between a retracted position and a deployed position. The sample capture device 10, 200 can further comprise means for effecting movement of the at least one capture assembly 50, 250 about and between the retracted position and the deployed position.

### Mucosal Tissue Sample Capture Cevice Having Single Central Wire

In one aspect, and with reference to FIGS. 1-8, the sample capture device 10 can comprise an elongate conduit 14 surrounding the central axis 12. In this aspect, the elongate conduit 14 can define a central bore 16 and have an outer surface 18, a first end 20, and an opposed, insertional end 22. Optionally, it is contemplated that the outer surface 18 of the elongate conduit 14 can have a plurality of spaced markings 13 configured to permit a user to precisely monitor the length of the portion of the sample capture device 10 that is inserted into the lumen of the esophagus of the subject. In exemplary aspects, the plurality of spaced markings 13 can be spaced apart from one another by 1 cm, and each marking can be labeled as a corresponding distance (e.g., 1 cm, 2 cm, 3 cm, etc.). It is contemplated that the elongate conduit 14 can have an outer diameter ranging from about 2.0 mm to about 7.0 mm. In exemplary aspects, it is contemplated that the outer diameter of the elongate conduit 14 can range from about 2.0 mm to about 2.5 mm. It is further contemplated that the elongate conduit can have an inner diameter ranging from about 1 mm to about 6 mm. In exemplary aspects, it is contemplated that the inner diameter of the elongate conduit 14 can range from about 1.25 mm to about 1.75 mm. In exemplary aspects, the elongate conduit 14 can comprise one or more conventional materials for forming surgical instrument tubing, such as, for example and without limitation, the materials used to form cytology brush tubing. In further exemplary aspects, it is contemplated that at least a portion of the capture device 10, including, for example and without limitation, the outer surface 18 of the elongate conduit 14, can be coated with a protective coating configured to protect the mucosal surfaces of the esophagus from shear forces. In these aspects, it is contemplated that the protective coating can optionally comprise a layer of sugar that is configured to dissolve upon contact with the mucosal surfaces of the esophagus. It is further contemplated that the protective coating can optionally comprise a layer of viscous material, such as a layer of gel as is known in the art.

In another aspect, the sample capture device 10 can comprise a central wire 30 configured for selective movement relative to the central axis 12. In this aspect, it is contemplated that at least a portion of the central wire 30 can be positioned within the central bore 16 of the elongate conduit 14. It is contemplated that the central wire 30 can comprise stainless steel. Optionally, it is contemplated that the central wire 30 can be a solid wire. Alternatively, it is contemplated that the central wire 30 can be braided. In exemplary aspects, the central wire 30 can have an outer diameter ranging from about 0.20 mm to about 1.20 mm. In other exemplary aspects, the outer diameter of the central wire 30 can range from about 0.60 mm to about 0.70 mm.

In an additional aspect, the sample capture device 10 can comprise at least one sheath 40 operatively coupled to the outer surface 18 of the elongate conduit 14. In this aspect, it is contemplated that each sheath 40 can have an outer diameter ranging from about 1.5 mm to about 7 mm. In exemplary aspects, the outer diameter of each sheath 40 can range from about 2.70 mm to about 2.80 mm. It is further contemplated that each sheath 40 can have an inner diameter ranging from about 1 mm to about 6 mm. In exemplary aspects, the inner diameter of each sheath 40 can range from about 2.20 mm to about 2.40 mm.

In a further aspect, the sample capture device 10 can comprise at least one capture assembly 50 operatively coupled to the central wire 30. In this aspect, it is contemplated that selective axial movement of the central wire 30 can be configured to effect axial and radial movement of each capture assembly 50 of the at least one capture assembly. It is further contemplated that each capture assembly 50 of the at least one capture assembly can be axially moveable about and between an enclosed position and an open position. In the enclosed position, it is contemplated that each capture assembly 50 of the at least one capture assembly can be positioned within a respective sheath 40 of the at least one sheath. In the open position, it is contemplated that each capture assembly 50 of the at least one capture assembly can be axially advanced beyond a respective sheath 40 such that the capture assembly is positioned outside the sheath.

In exemplary aspects, each capture assembly 50 of the at least one capture assembly can comprise a plurality of buckling elements 52. In these aspects, the plurality of buckling elements 52 can be azimuthally spaced relative to the central axis 12. For example, the plurality of buckling elements 52 can be substantially equally spaced from the central axis 12 but positioned at distinct, azimuthally spaced locations. It is contemplated that each buckling element 52 of the plurality of buckling elements can have a first end 54 and an opposed second end 56. In one aspect, each buckling element 52 of the plurality of buckling elements can have an inner surface 58 (closest to the central axis 12) and an outer surface 59 (farthest from the central axis). In exemplary aspects, each buckling element 52 can have a longitudinal length ranging from about 5 mm to about 60 mm. It is contemplated that each buckling element 52 can have a width ranging from about 0.25 mm to about 2.5 mm. In exemplary aspects, the width of each buckling element 52 can be about 1.0 mm. It is further contemplated that each buckling element 52 can have a thickness ranging from about 0.01 mm to about 0.4 mm. In exemplary aspects, the thickness of each buckling element 52 can be about 0.13 mm. In exemplary aspects, each buckling element can comprise one or more conventional thin elastic materials. For example, it is contemplated that each buckling element can comprise thin plastic that is optionally coated with a metallic coating.

Optionally, in further exemplary aspects, each buckling element 52 of the plurality of buckling elements can comprise at least one capture element 60 secured to the outer surface 59 of the buckling element such that the at least one capture element projects outwardly relative to the central axis 12. In these aspects, it is contemplated that the at least one capture element 60 can be configured to capture a mucosal tissue sample of the subject. In one aspect, each capture element 60 of the at least one capture element can comprise a sponge material. In another aspect, the at least one capture element 60 of each buckling element 52 can comprise a plurality of bristles. In exemplary aspects, each capture element 60 can have a longitudinal length ranging from about 0.5 cm to about 1.5 cm. In additional exemplary aspects, the longitudinal length of each capture element 60 can be about 1 cm. It is contemplated that each capture element 60 can have a width ranging from about 0.25 mm to about 2.5 mm. In exemplary aspects, the width of each capture assembly 60 can be about 1 mm. It is further contemplated that each capture element 60 can have a thickness ranging from about 0.1 mm to about 3 mm. In exemplary aspects, each capture element 60 can comprise a material selected from the group consisting of mascara brush bristles, lip gloss brush bristles, cotton, foam, hydrogels, polyelectrolyte hydrogels, cytology brush bristles, microfibers, synthetic fibers, expandable foam, and soft Velcro® as are known in the art. In other exemplary aspects, it is contemplated that each capture element 60 can comprise a membrane having a surface polyanionic charge to thereby increase the affinity of the membrane for the cationic eosinophilic granule proteins. In these aspects, it is contemplated that the membrane can comprise at least one of carboxylate and sulfate. However, it is contemplated that any anion that is capable of binding to the cationic granule proteins can be used. In still other exemplary aspects, it is contemplated that each capture element 60 can comprise a material having a hydrocarbon framework that permits binding of one or more acidic groups, including anions, such as, for example and without limitation, carbonates, sulfates, phosphates, nitrates, acetates, formats, or oxalates.

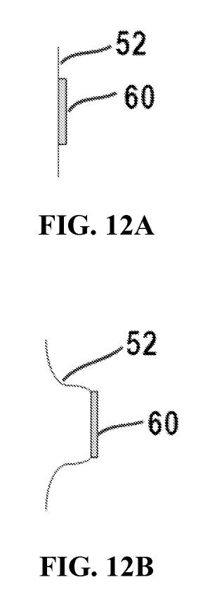

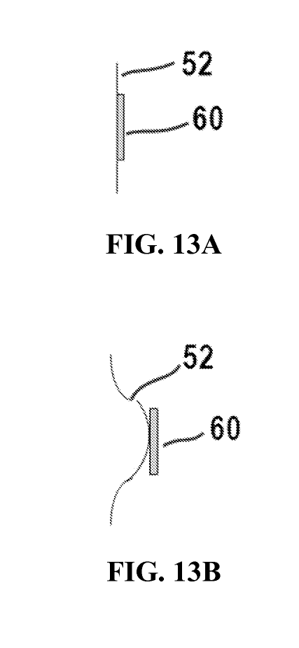

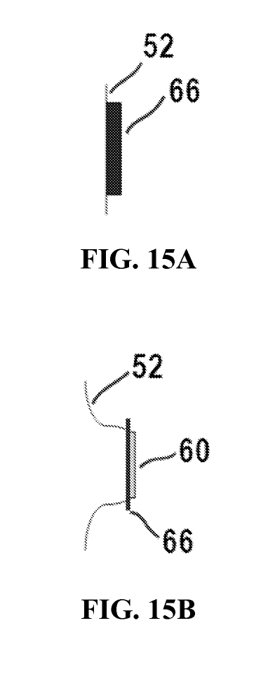

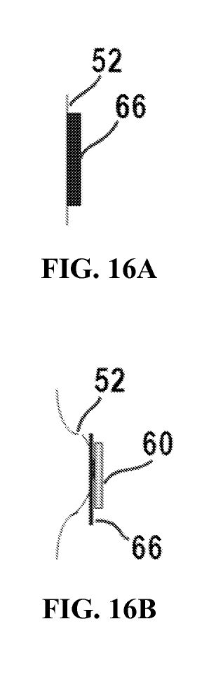

In exemplary aspects, and with reference to FIGS. 12 and 15, it is contemplated that each capture element 60 of each buckling element 52 can be substantially rigid (or stiff). It is contemplated that the rigid capture elements 60 can have sufficient rigidity to cause the buckling elements 52 to remain substantially flat during use, thereby maximizing the surface area of the capture element that is exposed to the mucosal surface of the esophagus (and increasing the amount of material collected by the capture device 10). Alternatively, and with reference to FIGS. 13 and 16, it is contemplated that each capture element 60 of each buckling element can be substantially limp (or elastic). It is contemplated that the limp capture elements 60 can be configured to bend to conform to the shape of the esophagus, thereby maximizing contact with the mucosal surface of the esophagus (and increasing the amount of material collected by the capture device 10).

In additional aspects, each capture assembly 50 of the at least one capture assembly can comprise a first crimping element 70 operatively coupled to the first ends 54 of the plurality of buckling elements 52 and to the central wire 30. In further aspects, each capture assembly 50 of the at least one capture assembly can comprise a second crimping element 72 operatively coupled to the second ends 56 of the plurality of buckling elements 52 such that the central wire 30 is axially moveable relative to the second crimping element. It is contemplated that the first and second crimping elements 70, 72 can have an outer diameter ranging from about 0.3 mm to about 7.0 mm. In exemplary aspects, the first and second crimping elements 70, 72 can have an outer diameter ranging from about 1.8 mm to about 2.3 mm. It is further contemplated that the first and second crimping elements 70, 72 can have an inner diameter ranging from about 1 mm to about 1.5 mm. In exemplary aspects, the first and second crimping elements 70, 72 can comprise electrical shrink wrap or like materials. In exemplary aspects, it is contemplated that the buckling elements 52 can be glued to the first and second crimping elements 70, 72.

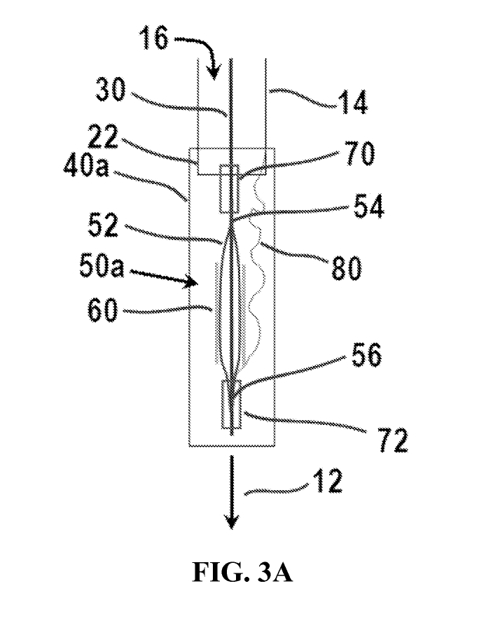

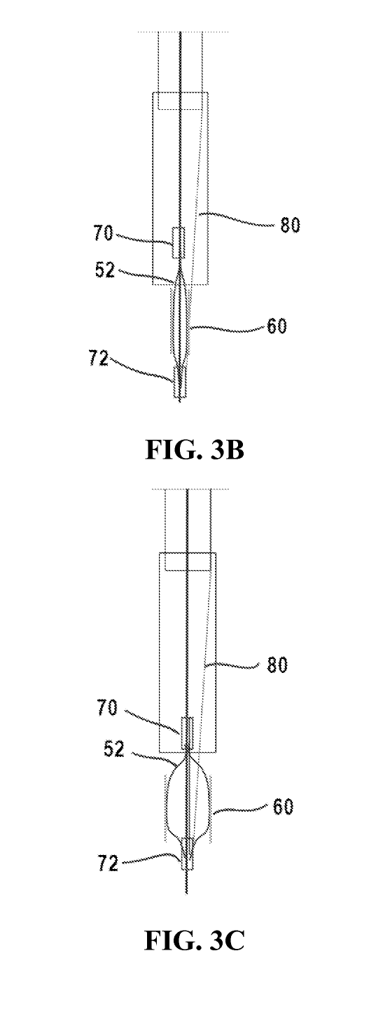

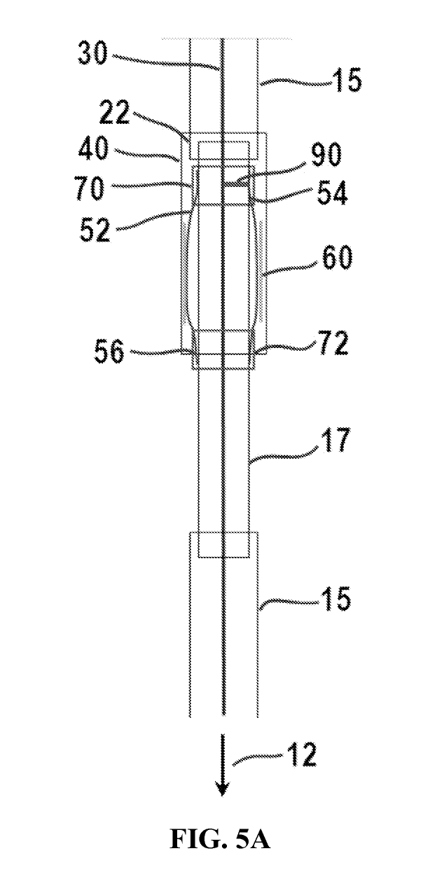

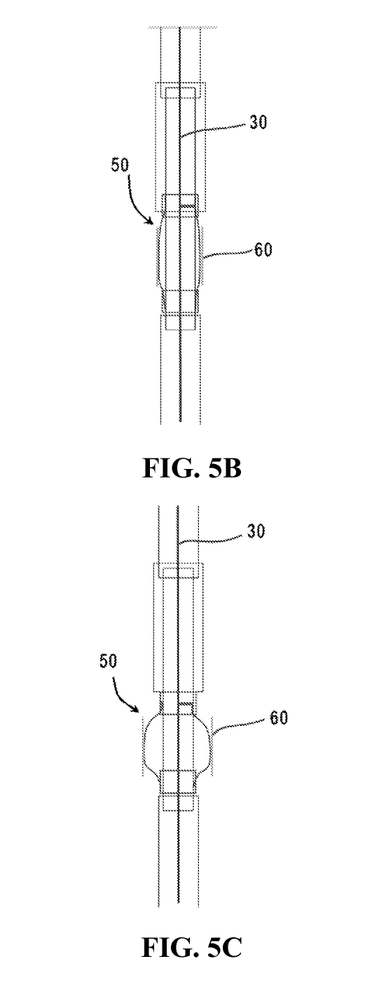

It is contemplated that each capture assembly 50 of the at least one capture assembly can be radially moveable about and between a retracted position and a deployed position. In the deployed position, it is contemplated that the first and second crimping elements 70, 72 of each capture assembly 50 can compress the plurality of buckling elements 52 of the capture assembly such that the plurality of buckling elements extend outwardly relative to the central axis 12. As shown in FIGS. 3 and 5, the operative diameter of each capture assembly 50 is greater in the deployed position than in the retracted position.

When the capture assembly 50 is positioned in the deployed position, it is contemplated that the plurality of buckling elements 52 can be positioned proximate esophageal tissue of the subject. It is further contemplated that, when the plurality of buckling elements 52 are positioned in the deployed position, the sample capture device 10 can be configured for selective axial movement relative to the central axis 12 and/or rotation about the central axis such that contact between the esophageal tissue of the subject and the capture elements 60 is maximized. It is further contemplated that selected sequences of axial movement and/or rotation of the sample capture device 10 can be repeated as necessary to capture a desired amount of mucosal tissue.

In exemplary aspects, each capture element 60 of each buckling element 52 can be detachably secured to the outer surface 59 of the buckling element. In these aspects, it is contemplated that the capture elements 60 of each buckling element 52 can be selectively detached from the buckling elements 52 and transported for ex vivo analysis of a recovered tissue sample as disclosed herein. In exemplary aspects, the capture elements 60 can be glued to respective buckling elements 52. It is further contemplated that the capture elements 60 can be secured to respective buckling elements 52 using small filaments. Alternatively, however, it is contemplated that each capture element 60 of each buckling element 52 can be integrally formed with the buckling element.

Optionally, in various aspects, at least a portion of each capture element 60 of the plurality of buckling elements 52 can be coated with a material configured to promote adhesion of a mucosal tissue sample of the subject to the capture element. In exemplary aspects, each capture element 60 can be coated with one or more materials selected from the group consisting of heparin, antibodies, gold sheets, and gold nanoparticles. In one aspect, it is contemplated that a heparin coating can be applied to an outer surface of each capture element 60 using conventional methods, such as, for example and without limitation, the methods disclosed in U.S. Pat. No. 4,871,357, which is incorporated herein by reference in its entirety. Other exemplary heparin-coating techniques are disclosed by Hsu (1991). In this aspect, it is contemplated that the heparin can be applied to the capture element 60 as the outermost layer of a multi-layer coating. Optionally, the heparin layer can be charged. In another aspect, an antibody for MBP-1 and/or an antibody for EPO can be attached to the capture element 60 using conventional methods. In an additional aspect, a gold sheet can be applied to the capture element to trap MBP-1. In this aspect, it is contemplated that the gold sheet can be applied to the capture element using conventional methods, including, for example and without limitation, atomic layer deposition, evaporation, sputter coating, chemical vapor deposition, and the like. In a further aspect, gold nanoparticles can be applied to the capture element 60 to trap MBP-1. In one exemplary aspect, gold nanoparticles suspended in water (Ted Pella, Inc.) can be applied to the capture element 60, and the water can be evaporated such that the gold nanoparticles condense onto the surfaces of the capture element. However, it is contemplated that the gold nanoparticles can be applied to the capture element 60 using any conventional method.

Optionally, as shown in FIG. 18, the plurality of buckling elements 52 of each capture assembly 50 can be positioned in a substantially staggered configuration. For example, it is contemplated that at least one buckling element 52 of the plurality of buckling elements can be offset from another buckling element of the plurality of buckling elements relative to the central axis 12.

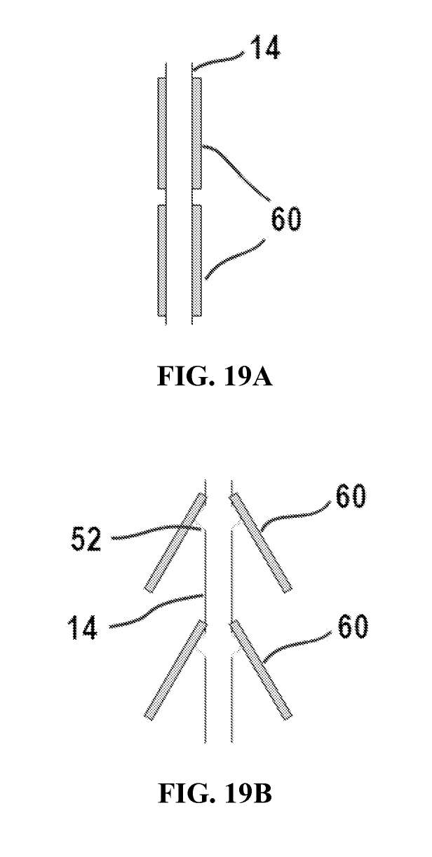

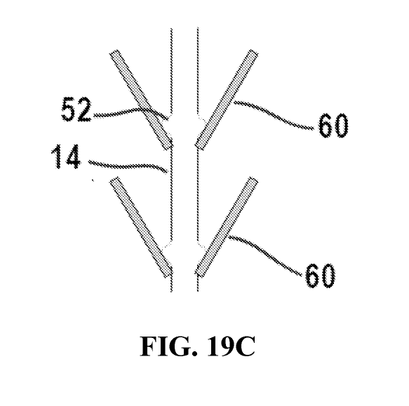

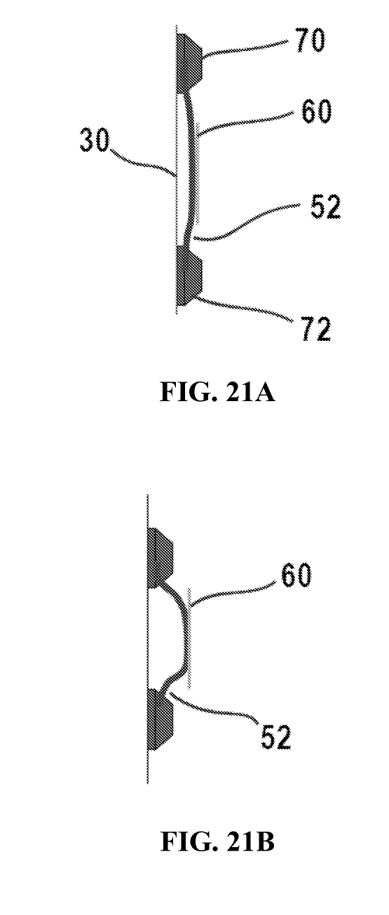

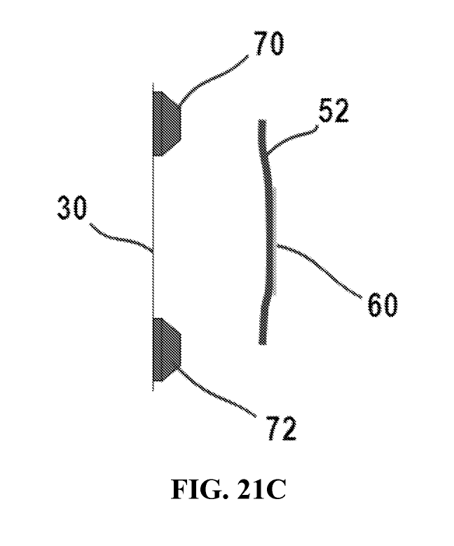

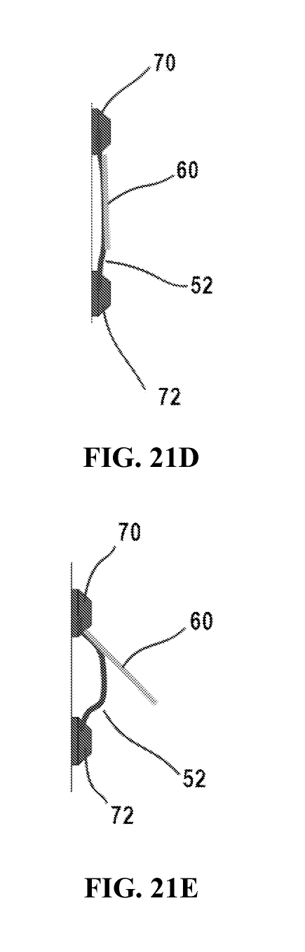

In exemplary aspects, as shown in FIG. 19, it is contemplated that the capture elements 60 can be larger than the buckling elements 52 to which they are secured. In these aspects, it is contemplated that each capture element 60 can be secured to a buckling element 52 at a first point proximate a crimping element and/or the elongate conduit 14 and at a second point that protrudes outwardly relative to the first point when the capture assembly is in the deployed position. Alternatively, it is contemplated that each capture element 60 can be secured to a buckling element 52 at a first point and to a portion of the elongate conduit 14 at a second point. As shown in FIGS. 19B-19C and 21D-21E, it is contemplated that, in these configurations, when the buckling elements buckle (and the capture assembly is in the deployed position), the capture elements can be configured to angle outwardly, thereby maximizing tissue collection. It is further contemplated that the capture elements can be angularly oriented relative to adjacent capture elements as measured relative to a plane transverse to the central axis 12.

In additional exemplary aspects, and with reference to FIG. 21, it is contemplated that the first and second crimping elements 70, 72 can each define one or more respective pockets, with the pockets of the first crimping element being configured to securely receive the first ends of the buckling elements 52 and the pockets of the second crimping element being configured to securely receive the second ends of the buckling elements. In use, it is contemplated that as the crimping elements 70, 72 converge toward one another, the buckling elements 52 can be held in place by their own tension. It is further contemplated that if a force perpendicular and away from the central axis 12 is applied, then both ends of the buckling elements 52 can be pulled out of their pockets such that the capture element 60 can be assayed as disclosed herein. The removal of the buckling elements 52 from the pockets of the crimping elements 70, 72 is depicted in FIGS. 21A-21C.

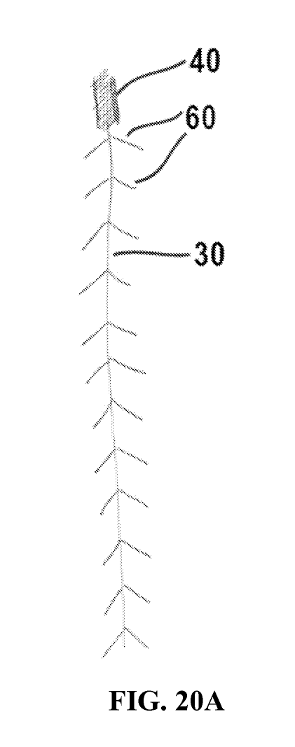

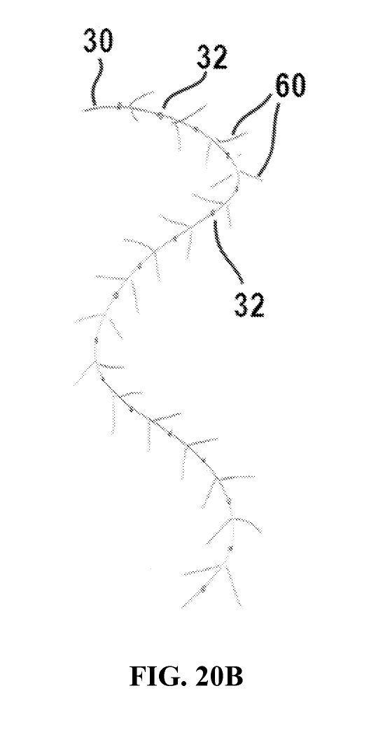

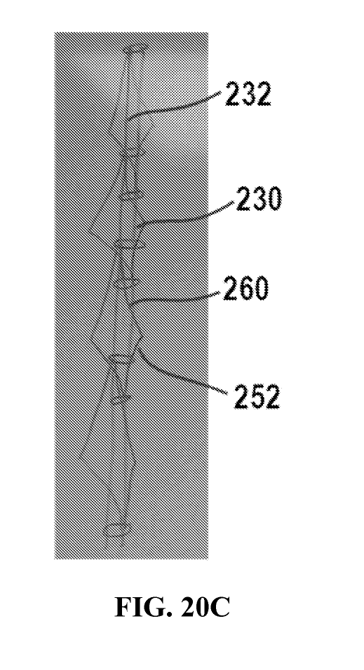

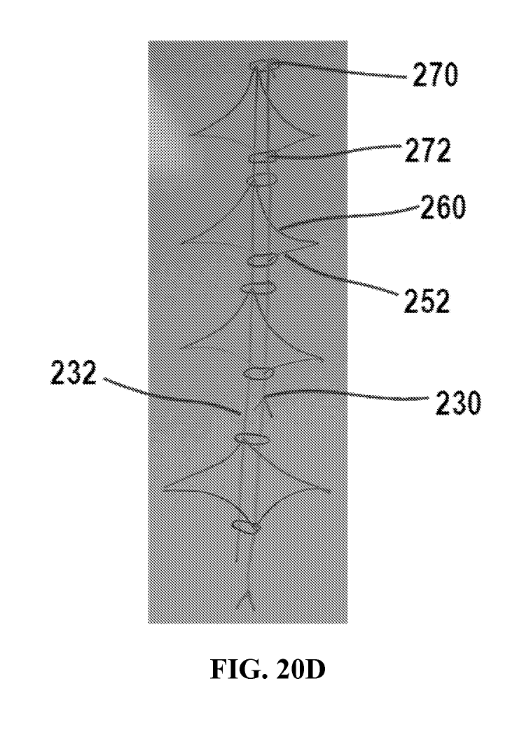

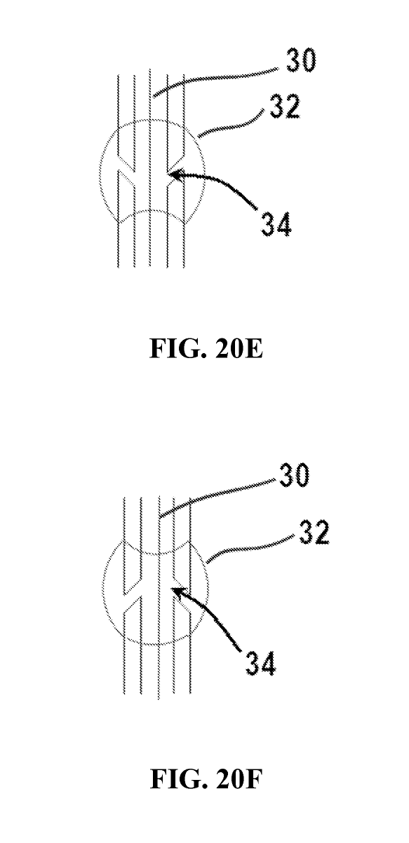

In still further exemplary aspects, and with reference to FIG. 20, it is contemplated that the capture elements 60 can optionally be secured directly to the central wire 30. In these aspects, it is contemplated that the capture device 10 can optionally comprise a plurality of joints 34. Optionally, each joint 34 of the plurality of joints can be surrounded by a joint support element 32. As shown in FIGS. 20A-20B, it is contemplated that the plurality of joints 34 can be formed in the central wire 30, with a plurality of joint support elements 34 being secured to the central wire 30 proximate the joints. Alternatively, as shown in FIGS. 20E-20F, it is contemplated that the plurality of joints 32 can be formed in the elongate conduit, with a joint support element 34 surrounding each joint 32 and being secured to the elongate conduit 14 proximate a respective joint. In exemplary aspects, it is contemplated that the joint support elements 34 can secure sequential portions of the central wire 30 and/or elongate conduit 14 together.

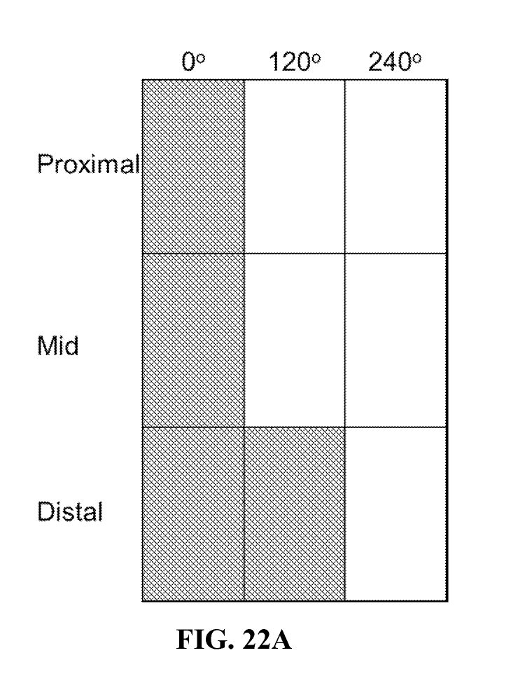

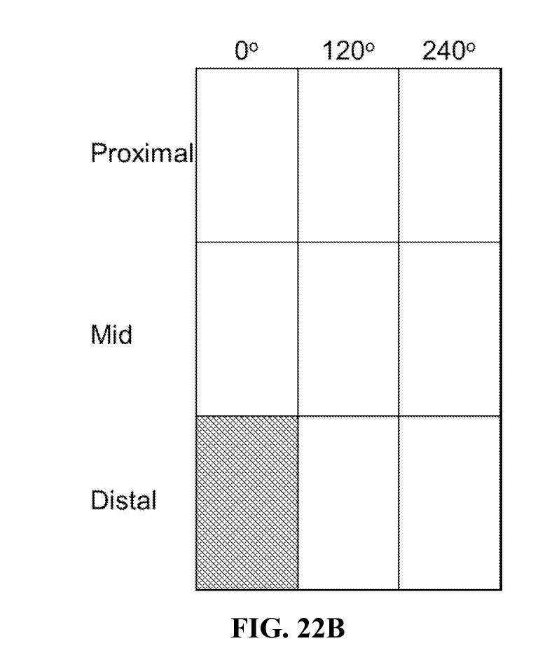

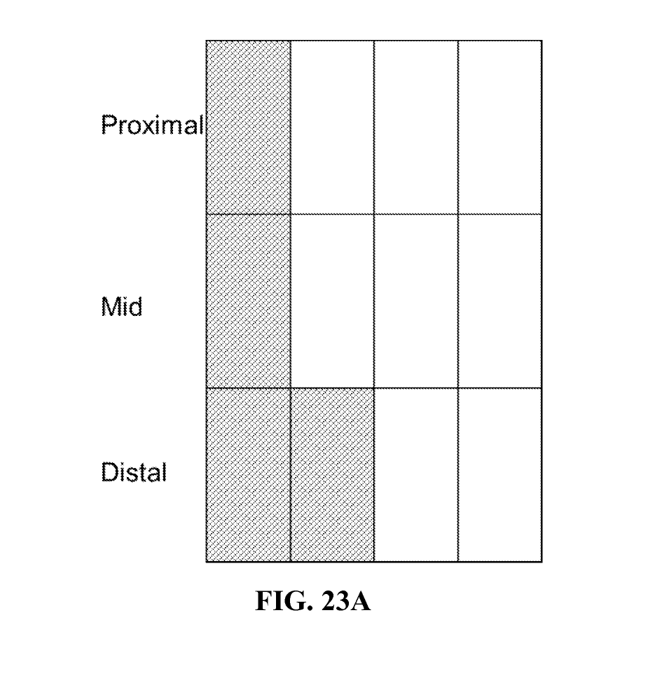

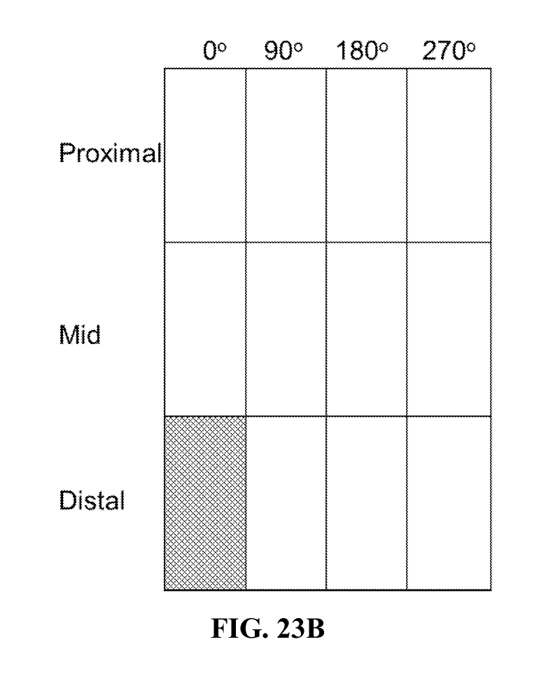

In one exemplary aspect, the plurality of buckling elements 52 can comprise three buckling elements. In this aspect, the plurality of buckling elements 52 can be substantially equally azimuthally spaced relative to the central axis 12, such as, for example and without limitation, at about 0 degrees, about 120 degrees, and about 240 degrees relative to the central axis 12. In another exemplary aspect, the plurality of buckling elements 52 can comprise four buckling elements. In this aspect, the plurality of buckling elements 52 can be substantially equally azimuthally spaced relative to the central axis 12, such as, for example and without limitation, at about 0 degrees, about 90 degrees, about 180 degrees, and about 270 degrees relative to the central axis 12. In another exemplary aspect, the at least one capture assembly 50 can comprise three capture assemblies. In this aspect, it is contemplated that, in the deployed position, the three capture assemblies can be spaced apart by about 8 cm relative to adjacent capture assemblies, thereby covering 24 cm, the approximate full length of an adult esophagus. Although sample capture devices having three capture assemblies are specifically described herein, it is contemplated that the at least one capture assembly 50 can comprise any number of capture assemblies, such as, for example and without limitation, 2, 4, 5, 6, 7, 8, 9, 10, 11, 12, 13, 14, 15, 16, 17, 18, 19, 20, 21, 22, 23, or 24 capture assemblies, with the capture assemblies being substantially equally axially spaced relative to the central axis. It is further contemplated that the capture assemblies 50 can be substantially equally axially spaced relative to the central axis 12. In still further exemplary aspects, the plurality of buckling elements 52 can comprise three buckling elements, and the at least one capture assembly 50 can comprise three capture assemblies as disclosed herein, thereby forming a 3×3 array of buckling elements and/or capture elements 60. In still further exemplary aspects, the plurality of buckling elements 52 can comprise four buckling elements, and the at least one capture assembly 50 can comprise three capture assemblies as disclosed herein, thereby forming a 3×4 array of buckling elements and/or capture elements 60. FIGS. 22 and 23 display exemplary comparison maps of esophageal disease obtained using 3×3 and 3×4 arrays of buckling elements, respectively. FIGS. 22A and 23A display an initial disease map, whereas FIGS. 22B and 23B display a later disease map (following disease progression and/or treatment). The three capture assemblies are respectively labeled as “proximal,” “mid,” and “distal” capture assemblies.

In one aspect, and with reference to FIG. 3, the at least one sheath 40 can comprise a first sheath 40a secured to the insertional end 22 of the elongate conduit 14. In this aspect, the at least one capture assembly 50 can comprise a first capture assembly 50a. In the enclosed position, the first capture assembly 50a can be positioned within the first sheath 40a. In another aspect, and as shown in FIG. 3, the sample capture device can further comprise a restraint wire 80 operatively coupled to and extending between the insertional end 22 of the elongate conduit 14 and the second crimping element 72 of the first capture assembly 50a such that the restraint wire limits the axial movement of the first capture assembly beyond the first sheath 40a. In use, when the first capture assembly 50a is positioned in the enclosed position, the restraint wire 80 is substantially limp. As the central wire 30 is pushed down, the buckling elements 52 are axially advanced with the central wire until the buckling elements are exposed (corresponding to the open position). When the restraint wire becomes taut, the first capture assembly 50a (and the buckling elements) are fully exposed and cannot be advanced any farther. With the first capture assembly 50a in the open position, the central wire 30 can be pushed down, thereby causing the buckling elements 52 to buckle, pucker, and/or bend such that the buckling elements extend outwardly relative to the central axis 12 (moving the first capture assembly from the retracted position to the deployed position). The central wire 30 can then be pulled up to return the first capture assembly 50a to the retracted position. The user can continue to pull up the central wire 30 to position the first capture assembly 50a within the first sheath 40a. In exemplary aspects, the restraint wire 80 can optionally comprise one or more fish line-type materials, such as, for example and without limitation, nylon, polyvinylidene fluoride (PVDF), polyethylene, Dacron®, ultra-high molecular weight polyethylene (UHMWPE), and the like.

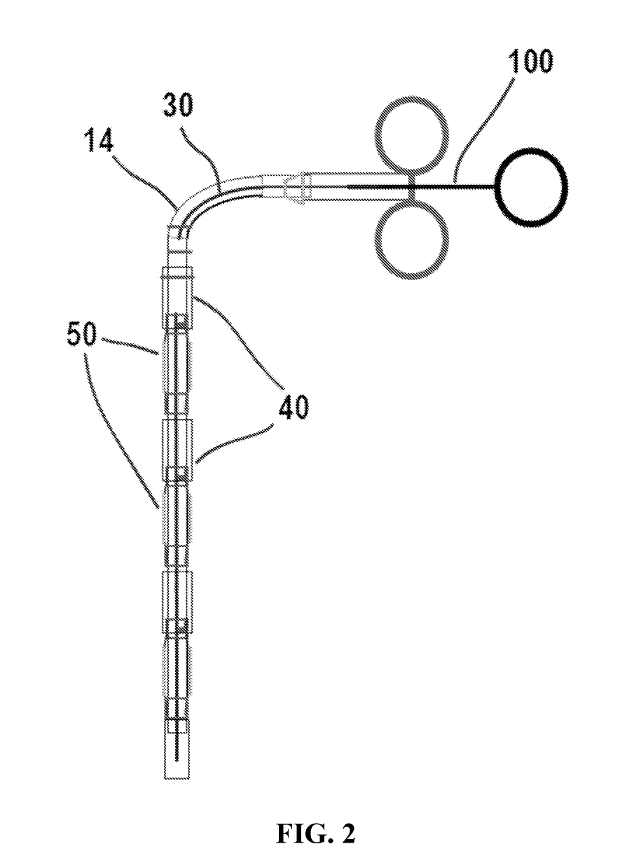

Optionally, in exemplary aspects, and as shown in FIGS. 2 and 5, the elongate conduit 14 can comprise a plurality of outer tubes 15 and at least one inner tube 17. In these aspects, each inner tube 17 of the at least one inner tube can be secured within and extend between sequential outer tubes 15 such that the at least one inner tube and the plurality of outer tubes cooperate to define the central bore 16 of the elongate conduit 14. It is contemplated that the plurality of outer tubes 15 can define the outer surface 18 of the elongate conduit 14. It is further contemplated that the inner and outer diameters and materials of the outer tubes 15 can correspond to the inner and outer diameters disclosed herein with respect to elongate conduit 14. However, it is contemplated that the inner tubes 17 can have an outer diameter ranging from about 0.7 mm to about 6 mm and. In exemplary aspects, the outer diameter of the inner tubes 17 can be about 1.5 mm. It is further contemplated that the inner tubes 17 can have an inner diameter ranging from about 0.5 mm to about 1 mm. In exemplary aspects, the inner diameter of the inner tubes 17 can be about 0.7 mm. In one aspect, as shown in FIG. 5, an inner tube 17 of the at least one inner tube can be received within the first and second crimping elements 70, 72 of a respective capture assembly 50 of the at least one capture assembly. In this aspect, it is contemplated that each capture assembly 50 can further comprise a restraint wire 90 operatively coupled to and extending between the first crimping element 70 of the capture assembly and the central wire 30. In exemplary aspects, the restraint wire 90 can optionally comprise one or more fish line-type materials, such as, for example and without limitation, nylon, polyvinylidene fluoride (PVDF), polyethylene, Dacron®, ultra-high molecular weight polyethylene (UHMWPE), and the like.

In an additional aspect, the plurality of outer tubes 15 can have an outer diameter, and the capture elements 60 of the plurality of buckling elements 52 of each capture assembly 50 of the at least one capture assembly can cooperate to define an operative diameter of the capture assembly. In this aspect, when the at least one capture assembly 50 is in a deployed position, the operative diameter of the at least one capture assembly can be greater than the outer diameter of the plurality of outer tubes 15 such that the plurality of outer tubes restrict axial movement of the at least one capture assembly.

As shown in FIG. 5, it is contemplated that the spacing between sequential outer tubes 15 of the elongate conduit 14 relative to the central axis 12 can correspond to portals for selective deployment of the capture assemblies 50. In use, each capture assembly 50 is initially positioned within a sheath 40 (the enclosed position). When the central wire 30 is pushed down, the capture assembly 50 is lowered from the sheath until the capture assembly (and all buckling elements 52) is exposed (positioned outside the sheath). The central wire 30 can be pushed down until the capture assembly 50 contacts an inner tube-outer tube junction as shown in FIG. 5B (the open position). With the capture assembly 50 in the open position, the central wire 30 is further advanced, causing the buckling elements 52 to buckle, pucker, and/or bend such that the buckling elements extend outwardly relative to the central axis 12 (moving the capture assembly from the retracted position to the deployed position). The central wire 30 can be pulled back (retracted) to return the capture assembly 50 to the retracted position. Further retraction of the central wire 30 can position the capture assembly 50 within the sheath 40 (in the enclosed position).

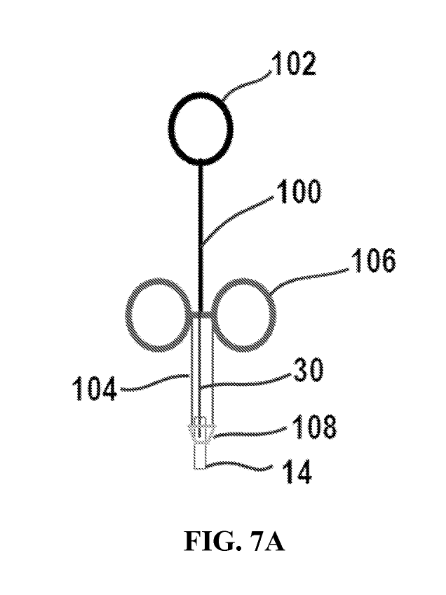

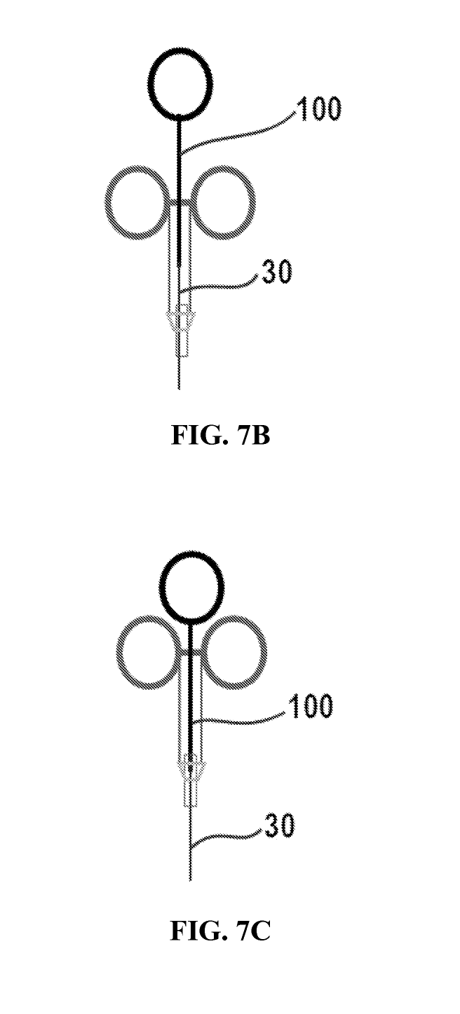

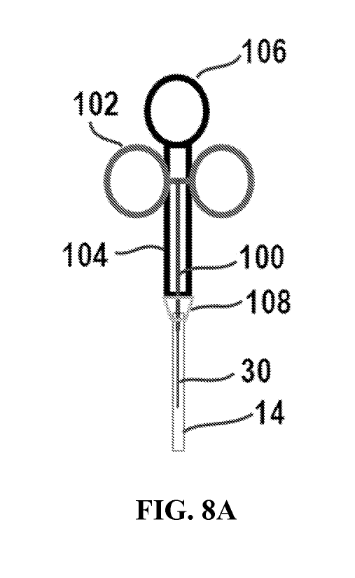

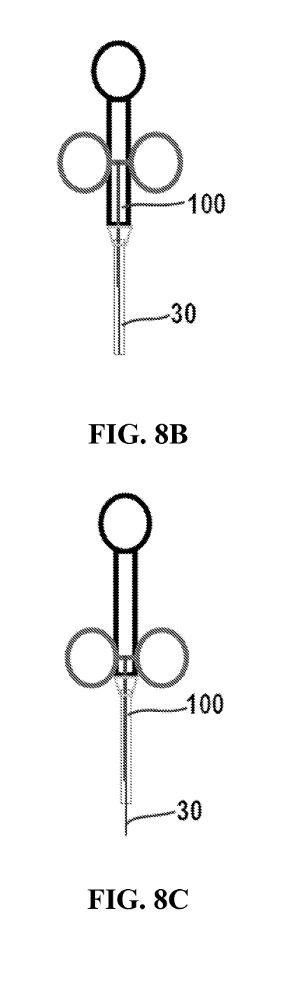

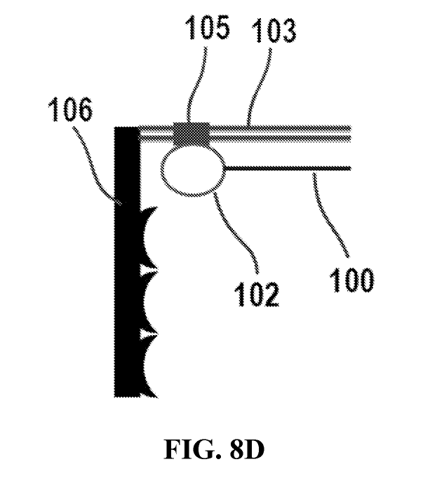

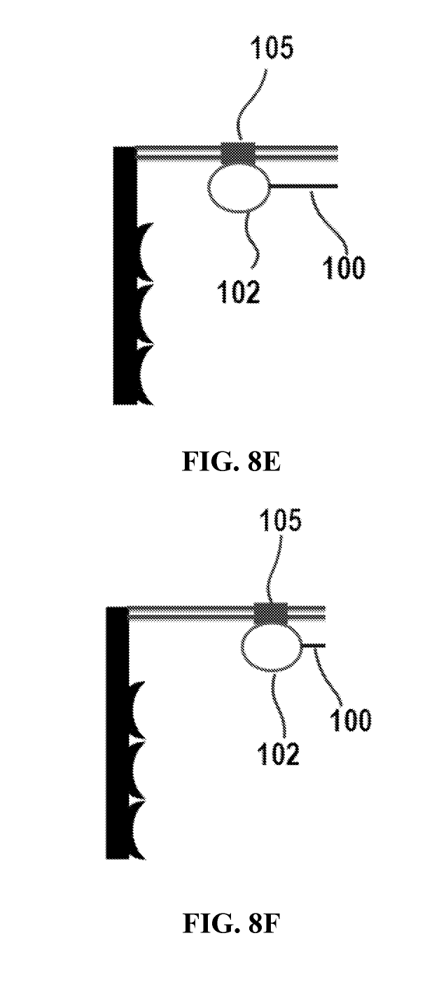

In additional exemplary aspects, and with reference to FIGS. 7 and 8, the sample capture device 10 can further comprise an actuator 100 configured for engagement by at least a portion of the hand of a user. In these aspects, the actuator 100 can comprise an engagement portion 102. Optionally, the engagement portion can define at least one opening configured to receive one or more fingers of a user. It is contemplated that the actuator 100 can be operatively coupled to the central wire 30 such that movement of the actuator relative to the central axis 12 effects a corresponding axial movement of the central wire. As shown in FIGS. 7A and 8A, in exemplary configurations, a fully retracted actuator 100 can correspond to the enclosed position of the at least one capture assembly 50. As shown in FIGS. 7B and 8B, in exemplary configurations, a partially depressed actuator 100 can correspond to the open position of the at least one capture assembly 50. As shown in FIGS. 7C and 8C, in exemplary configurations, a fully depressed actuator 100 can correspond to the deployed position of the at least one capture assembly 50.

As shown in FIGS. 7 and 8, it is contemplated that the actuator 100 can be provided as part of an assembly. In one aspect, the assembly can comprise a barrel 104 positioned in operative communication with the first end 20 of the elongate conduit 14 and configured to receive at least a portion of the actuator 100 such that the actuator can be selectively axially advanced within both the barrel and the elongate conduit. Optionally, in exemplary aspects, the assembly can further comprise one or more secondary engagement portions 106 configured for engagement with one or more fingers of a user. For example, in these aspects, it is contemplated that the secondary engagement portions 106 can define respective openings configured to receive one or more fingers of a user. In other aspects, as shown in FIGS. 8D-8F, it is contemplated that the secondary engagement portion 106 can comprise a series of finger grips. In additional aspects, the assembly can optionally comprise a coupler 108 configured to provide a secure, fluid-tight connection between the barrel 104 and the first end 120 of the elongate conduit 14.

Optionally, as shown in FIGS. 8D-8F, it is contemplated that the actuator assembly can further comprise a slider 105 operatively coupled to the engagement portion 102 of the actuator 100. It is contemplated that the slider 105 can be configured for sliding, axial movement relative to a track 103 selectively oriented relative to the actuator assembly. The sequential movement of the actuator 100 and slider 105 from the closed/retracted position of the capture assembly to the deployed position of the capture assembly is depicted in FIGS. 8D-8F.

Optionally, in one aspect, and with reference to FIGS. 1B, 4D, and 6D-6E, the sample capture device 10 can further comprise a pH probe 110 coupled to the insertional end 22 of the elongate conduit 14 and/or an insertional end of a selected sheath 40 positioned proximate the insertional end of the elongate conduit 14. It is contemplated that the pH probe can be configured to monitor the change in pH within the esophagus of the subject as the sample capture device 10 is advanced into the esophagus. It is further contemplated that the pH probe can permit a user to identify a selected location within the gastroesophageal tract of the subject, such as, for example and without limitation, the stomach of the subject or the gastroesophageal junction of the subject. Optionally, in exemplary aspects, the sample capture device 10 can further comprise a microchip 140 in electrical communication with the pH probe 110. In these aspects, the pH probe can be configured to produce an output indicative of the pH measured by the pH probe, and the microchip 140 can have a processor that is configured to identify the selected location within the gastroesophageal tract of the subject based on the output produced by the pH probe. It is contemplated that the processor of the microchip 140 can be electrically coupled to a light such that the light is selectively activated when the pH probe detects an acidic pH (corresponding to the pH probe reaching the stomach of the subject). It is further contemplated that the light can be a LED light. It is still further contemplated that the pH probe can be positioned in electrical communication with the microchip using any conventional wired or wireless means. Where wired communication means are used, it is contemplated that one or more wires connected between the pH probe and the microchip can pass entirely within the elongate conduit 14 or only partially within the elongate conduit (such that a portion of the one or more wires is positioned external to the elongate conduit 14).

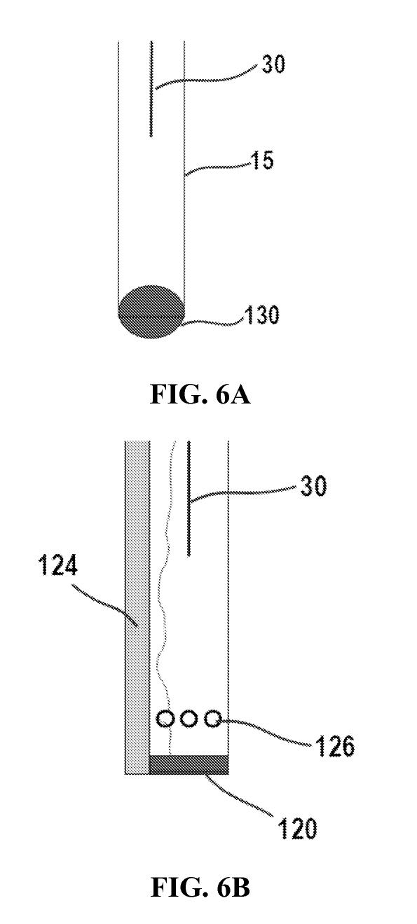

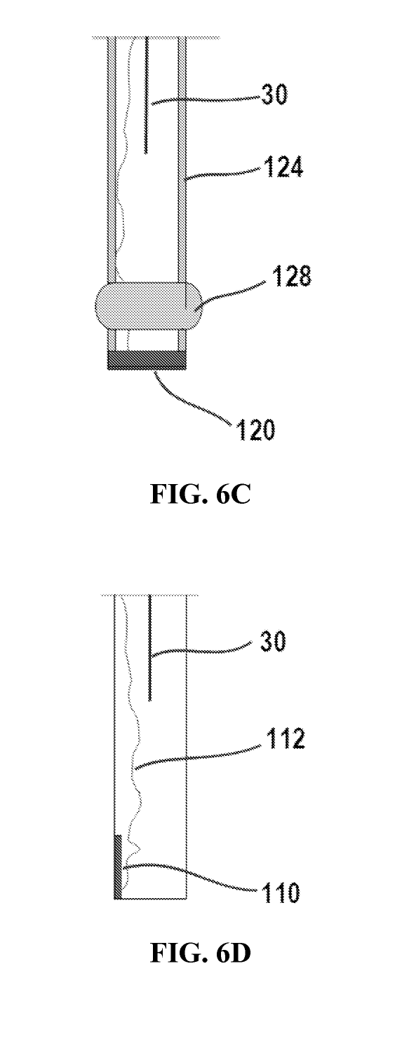

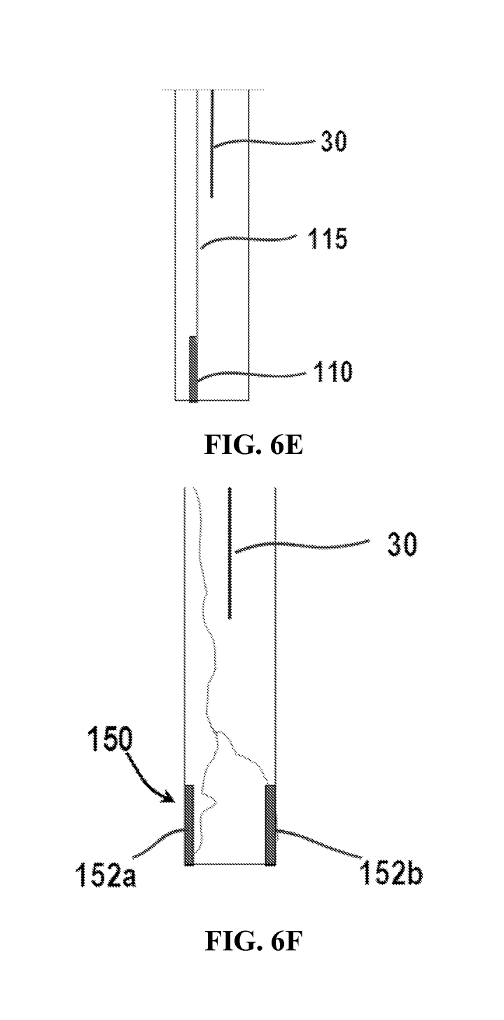

Optionally, as shown in FIG. 6E, rather than being coupled to the elongate conduit 14, the pH probe 110 can be coupled to a secondary wire 115 that is selectively axially moveable relative to the central axis 12 of the sample capture device 10. It is contemplated that the secondary wire 115 can be selectively axially moved such that the pH probe 110 is positioned within the elongate conduit 14 and/or within a sheath 40 of the sample capture device 10. It is further contemplated that the secondary wire 115 can be selectively axially moved such that the pH probe 110 is advanced beyond—and positioned outside—the elongate conduit 14 and/or within a sheath 40 of the sample capture device 10.

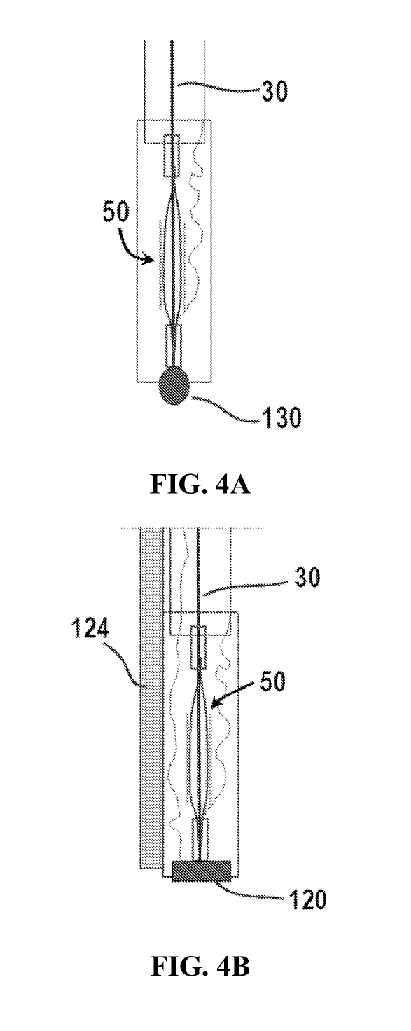

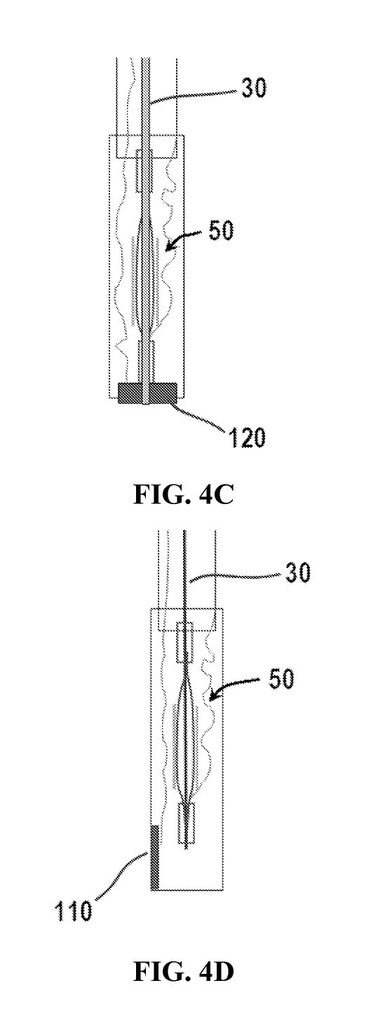

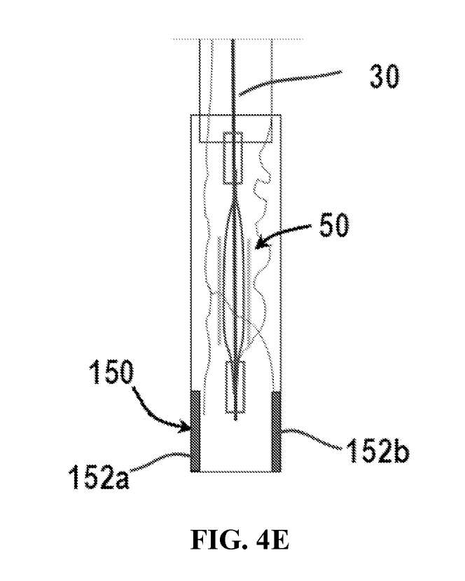

In another optional aspect, as shown in FIGS. 4B-4C and 6B-6C, the sample capture device 10 can further comprise imaging means, such as for example and without limitation, a camera 120 (as shown) or a fiber optic cable having one or more optical fibers. The imaging means (e.g., camera 120) can be coupled to the insertional end 22 of the elongate conduit 14 (See FIG. 6B) or to an insertional end of a sheath 40 coupled to the insertional end 22 of the elongate conduit (See FIG. 4B). In this aspect, it is contemplated that the camera 120 can be a micro-camera as is known in the art. It is further contemplated that the imaging means can be configured to provide an image of the position of the insertional end 22 of the elongate conduit 14 or a sheath 40 within the subject. It is still further contemplated that the imaging means can be used to visually identify a selected location within the gastroesophageal tract of the subject, such as, for example and without limitation, the stomach of the subject or the gastroesophageal junction of the subject. In exemplary aspects, the imaging means can be positioned in electrical communication with a display means and/or image processing means. In these aspects, it is contemplated that the imaging means can be positioned in electrical communication with the display means and/or image processing means using conventional wired or wireless transmission mechanisms. In other exemplary aspects, it is contemplated that the sample capture device 10 can further comprise a light source in communication with the imaging means. In these aspects, it is contemplated that the sample capture device 10 can define a light channel 124 extending along at least a portion of the length of the sample capture device to permit transmission of light from the light source to the imaging means. In exemplary aspects, as shown in FIG. 4C, where the imaging means comprises a fiber optic cable, it is contemplated that the fiber optic cable can function as the central wire 30. Optionally, it is contemplated that the imaging means can comprise both a camera 120 and a fiber optic cable in communication with the camera. In other exemplary aspects, it is contemplated that the imaging means can be coupled to the elongate conduit 14 and/or a sheath 40 such that air flow is provided to the imaging source. It is contemplated that airflow can insulfate the esophagus such that a user of the device can determine the location of the device before it contacts the esophagus. In exemplary aspects, as shown in FIGS. 4B-4C, a gap between the imaging means and the inner surface of the elongate conduit 14 and/or sheath 40 can be provided such that an airflow is present proximate the imaging means. Alternatively, in other exemplary aspects, as shown in FIG. 6B, the capture device 10 can define at least one port 126 that is configured to provide airflow within the elongate conduit 14 and/or sheath 40 proximate the imaging means. In still other exemplary aspects, and with reference to FIG. 6C, the capture device 10 can comprise at least one balloon element 128 that surrounds the outer surface 18 of the elongate conduit 14 and/or an outer surface of a sheath 40 proximate the location of the imaging means. In these aspects, it is contemplated that the at least one balloon element 128 can be configured to expand the mucosal surface of the esophagus of the subject as needed such that the user of the capture device 10 can visualize where the device is within the esophagus of the subject.

Optionally, in another aspect, as shown in FIGS. 4A and 6A, the sample capture device 10 can further comprise a terminal bead 130. The terminal bead 130 can be coupled to the insertional end 22 of the elongate conduit (See FIG. 6A) or to an insertional end of a sheath 40 coupled to the insertional end 22 of the elongate conduit 14 (See FIG. 4A). In this aspect, it is contemplated that the terminal bead 130 can project from the insertional end 22 of the elongate conduit 14 or from the insertional end of the sheath 40 to thereby protect the conduit and/or sheath and to assist with controlled insertion of the sample capture device 10 by providing additional weight to the insertional portion of the device.

Optionally, in another aspect, and as shown in FIGS. 4E and 6F, the sample capture device can further comprise a conductivity probe 150 having at least first and second leads 152a, 152b. In this aspect, it is contemplated that the conductivity probe 150 can be configured to detect changes in resistance between the first and second leads 152a, 152b. It is further contemplated that the changes in resistance detected by the conductivity probe 150 can be indicative of the presence of gastric liquid proximate the conductivity probe. In exemplary aspects, the conductivity probe 150 can be coupled to the insertional end 22 of the elongate conduit (See FIG. 6F) or to an insertional end of a sheath 40 coupled to the insertional end 22 of the elongate conduit 14 (See FIG. 4E).

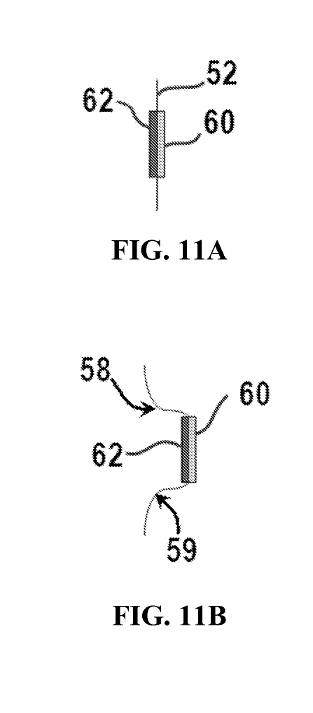

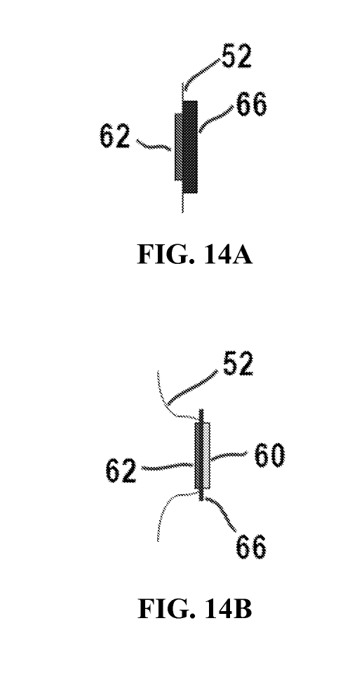

In still another optional aspect, as shown in FIGS. 11 and 14, it is contemplated that each buckling element 52 of the plurality of buckling elements can comprise at least one shim element 62 secured to the inner surface 58 of the buckling element. In this aspect, each shim element 62 of the at least one shim element can be secured in opposition to one or more capture elements 60 of the at least one capture element. It is contemplated that the at least one shim element 62 can be configured to flatten the at least one capture element.

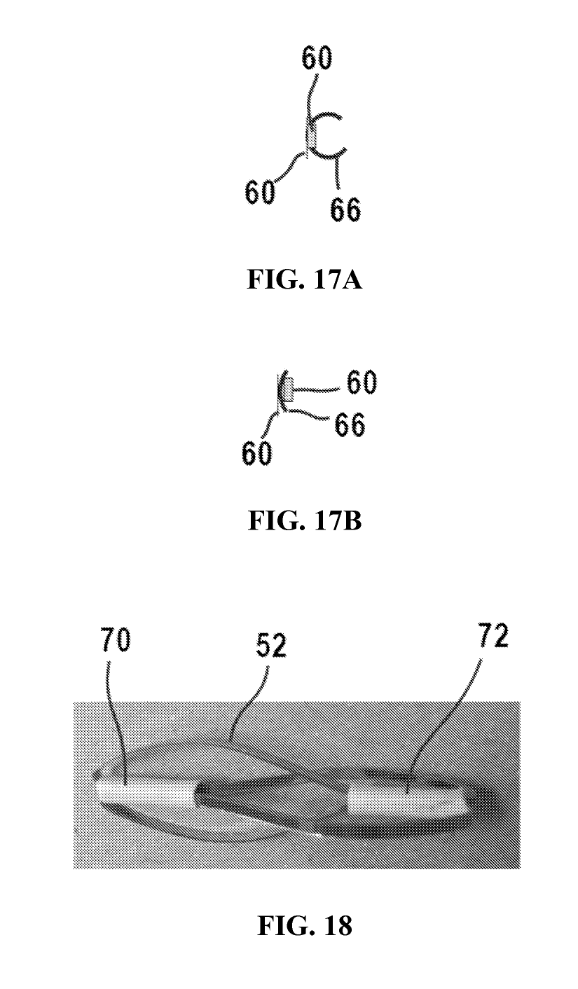

Optionally, and with reference to FIGS. 14-17, in additional exemplary aspects, each buckling element 52 of the plurality of buckling elements of each capture assembly 50 can comprise at least one enclosing element 66 secured to the outer surface 59 of the buckling element. In these aspects, the at least one enclosing element 66 can be deformable between a closed position and an open position. It is contemplated that the at least one enclosing element 66 can be operatively coupled to the buckling element 52 such that when the capture assembly 50 is in the retracted position the at least one enclosing element is in the closed position and when the capture assembly is in the deployed position the at least one enclosing element is in the open position. It is further contemplated that, in the closed position, each enclosing element 66 can shield at least a portion of the capture element 50 from the external environment. In exemplary aspects, in the closed position, each enclosing element 66 can substantially envelop a capture element 50 of the at least one capture element. It is still further contemplated that, in the open position, each enclosing element 66 can be retracted to expose at least a portion of a capture element 50 of the at least one capture element. In use, it is contemplated that the at least one enclosing element 66 can be configured to protect the capture elements 60 until deployment of the capture elements for recovery of tissue samples. In exemplary aspects, and as shown in FIG. 14, it is contemplated that each buckling element 52 of the plurality of buckling elements of each capture assembly 50 can be provided with both a shim element 62 and an enclosing element 66.

In exemplary aspects, it is contemplated that one or more of the components of the disclosed sample capture device 10 can be provided in the form of a kit.

### Mucosal Tissue Sample Capture Device Having First and Second Central Wires

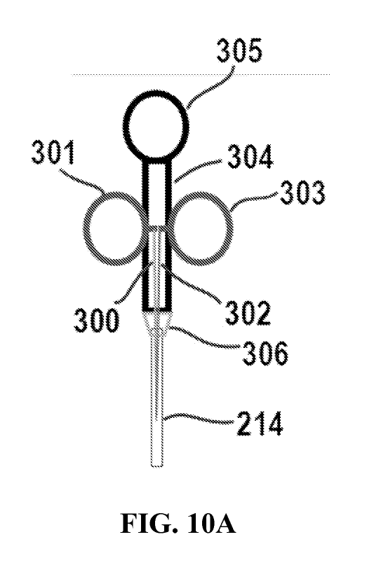

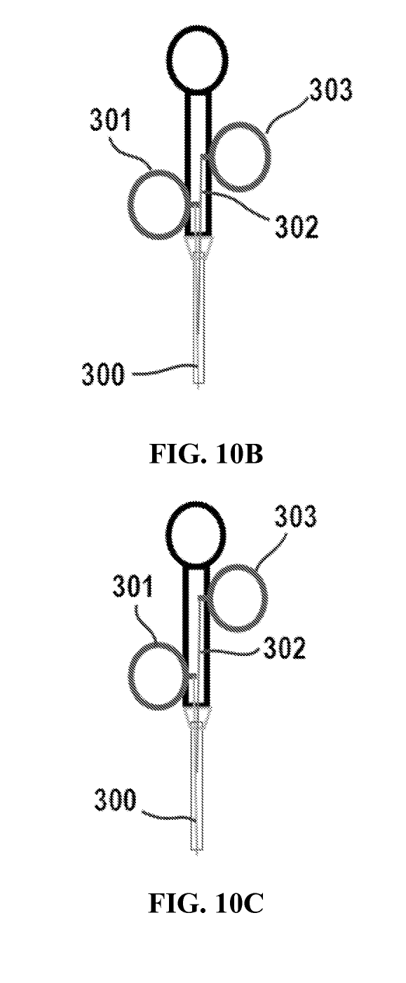

In one aspect, and with reference to FIGS. 9-10, the sample capture device 200 can comprise first and second central wires 230, 232 configured for selective movement relative to the central axis 202. Optionally, it is contemplated that the sample capture device 200 can comprise an elongate conduit (as disclosed herein with respect to sample capture device 10) that receives at least a portion of the first and second central wires 230, 232. It is further contemplated that the elongate conduit of the sample capture device 200 can have an outer surface with a plurality of spaced markings as disclosed herein with respect to sample capture device 10. It is contemplated that the dimensions and materials of the first and second central wires 230, 232 can match the disclosed dimensions and materials of the central wire disclosed herein with respect to sample capture device 10. In further exemplary aspects, it is contemplated that at least a portion of the capture device 200, including, for example and without limitation, the outer surface of the elongate conduit, can be coated with a protective coating configured to protect the mucosal surfaces of the esophagus from shear forces. In these aspects, it is contemplated that the protective coating can optionally comprise a layer of sugar that is configured to dissolve upon contact with the mucosal surfaces of the esophagus. It is further contemplated that the protective coating can optionally comprise a layer of viscous material, such as a layer of gel as is known in the art.

In another aspect, the sample capture device 200 can comprise at least one sheath 240 operatively coupled to the first central wire 230. In this aspect, it is contemplated that selective movement of the first central wire 230 can be configured to effect movement of each sheath 240 of the at least one sheath about and between a closed position and an open position. In exemplary aspects, each sheath 240 can have an outer diameter ranging from about 4 mm to about 6 mm and, more preferably, being about 5 mm. In these aspects, it is contemplated that each sheath 240 can have an inner diameter ranging from about 4 mm to about 5 mm and, more preferably, being about 4.5 mm. In exemplary aspects, the at least one sheath 240 can comprise electrical shrink wrap or like materials.

In an additional aspect, the sample capture device 200 can comprise at least one capture assembly 250 operatively coupled to the second central wire 232. In this aspect, it is contemplated that selective movement of the second central wire 232 can be configured to effect movement of each capture assembly 250 of the at least one capture assembly about and between a retracted position and a deployed position. As shown in FIG. 9, the operative diameter of each capture assembly 250 is greater in the deployed position than in the retracted position.

In the closed position of the at least one sheath 240, it is contemplated that each capture assembly 250 of the at least one capture assembly can be positioned within a respective sheath of the at least one sheath. In the open position of the at least one sheath 240, it is contemplated that each sheath of the at least one sheath can be axially advanced relative to a respective capture assembly 250 such that the capture assembly is positioned outside the sheath.

In exemplary aspects, each capture assembly 250 of the at least one capture assembly can comprise a plurality of buckling elements 252. In these aspects, it is contemplated that the plurality of buckling elements 252 can be azimuthally spaced relative to the central axis 202. It is further contemplated that each buckling element 252 can have a first end 254 and an opposed second end 256. It is still further contemplated that each buckling element 252 can have an inner surface 258 (closest to the central axis) and an outer surface 259 (farthest from the central axis). It is contemplated that the dimensions and materials of each buckling element 252 can correspond to the dimensions and materials of the buckling elements disclosed herein with respect to sample capture device 10.

In one aspect, each capture assembly 250 of the at least one capture assembly can further comprise a first crimping element 270 operatively coupled to the first ends 254 of the plurality of buckling elements 252 such that the first and second central wires 230, 232 are axially moveable relative to the first crimping element. In another aspect, each capture assembly 250 of the at least one capture assembly can further comprise a second crimping element 272 operatively coupled to the second ends 256 of the plurality of buckling elements 252 and the second central wire 232. In this aspect, it is contemplated that the first central wire 230 can be axially moveable relative to the second crimping element 272. In the deployed position, it is contemplated that the second crimping element 272 can compress the plurality of buckling elements 252 of each capture assembly 250 such that the plurality of buckling elements extend outwardly relative to the central axis 202. It is contemplated that the dimensions and materials of the first and second crimping elements 270, 272 can correspond to the dimensions and materials of the first and second crimping elements disclosed herein with respect to sample capture device 10.

In use, when each capture assembly 250 is positioned within a respective sheath 240 (in the closed position), it is contemplated that the first central wire 230 can be pushed down such that each sheath 240 is axially advanced, thereby exposing at least a portion of each capture assembly (in the open position). With each capture assembly 250 positioned in the open position, the second central wire 232 can be pulled up (retracted) such that the first crimping element 270 of each capture assembly contacts an adjacent sheath 240, and the first and second crimping elements 270, 272 cause the buckling elements 252 to buckle, pucker, and/or bend such that they extend outwardly relative to the central axis 202 (moving from the retracted position to the deployed position). From the deployed position, the second central wire 232 can be pushed downwardly (axially advanced) and the first central wire 230 can be pulled upwardly (retracted) to sequentially return the capture 250 to the retracted position and return the sheaths 240 to the closed position.

In exemplary aspects, each buckling element 252 of the plurality of buckling elements can comprise at least one capture element 260 secured to the outer surface 259 of the buckling element such that the at least one capture element projects outwardly relative to the central axis 202. In these aspects, the at least one capture element 260 of each buckling element can be configured to capture a mucosal tissue sample of the subject. Optionally, in one aspect, it is contemplated that each capture element 260 of the at least one capture element can comprise a sponge material. In another optional aspect, it is contemplated that the at least one capture element 260 of each buckling element 252 can comprise a plurality of bristles. Optionally, in still further aspects, at least a portion of each capture element 250 of each buckling element 252 can be coated with a material configured to promote adhesion of a mucosal tissue sample of the subject to the capture element. It is contemplated that the capture elements 260 can have the same dimensions and comprise the same materials as the capture elements disclosed herein with respect to sample capture device 10.

When the capture assembly 250 is positioned in the deployed position, it is contemplated that the plurality of buckling elements can be positioned proximate esophageal tissue of the subject. It is further contemplated that, when the plurality of buckling elements are positioned in the deployed position, the sample capture device 200 can configured for selective axial movement relative to the central axis 202 and/or rotation about the central axis such that contact between the esophageal tissue of the subject and the capture elements 260 is maximized. It is further contemplated that selected sequences of axial movement and/or rotation of the sample capture device 200 can be repeated as necessary to capture a desired amount of mucosal tissue.

In exemplary aspects, each capture element 260 of each buckling element 252 can be detachably secured to the outer surface 259 of the buckling element. In these aspects, it is contemplated that the capture elements 260 of each buckling element 252 can be selectively detached from the buckling elements 252 and transported for ex vivo analysis of a recovered tissue sample as disclosed herein. Alternatively, however, it is contemplated that each capture element 260 of each buckling element 252 can be integrally formed with the buckling element.

In additional exemplary aspects, it is contemplated that each capture element 260 of each buckling element 252 can be substantially rigid. It is contemplated that the rigid capture elements 260 can have sufficient rigidity to cause the buckling elements 252 to remain substantially flat during use, thereby maximizing the surface area of the capture element that is exposed to the mucosal surface of the esophagus. Alternatively, it is contemplated that each capture element 260 of each buckling element 252 can be substantially limp. It is contemplated that the limp capture elements 260 can be configured to bend to conform to the shape of the esophagus, thereby maximizing contact with the mucosal surface of the esophagus.

In one exemplary aspect, the plurality of buckling elements 252 can comprise three buckling elements. In this aspect, the plurality of buckling elements 252 can be substantially equally azimuthally spaced relative to the central axis 202, such as, for example and without limitation, at about 0 degrees, about 120 degrees, and about 240 degrees relative to the central axis 202. In another exemplary aspect, the plurality of buckling elements 252 can comprise four buckling elements. In this aspect, the plurality of buckling elements 252 can be substantially equally azimuthally spaced relative to the central axis 202, such as, for example and without limitation, at about 0 degrees, about 90 degrees, about 180 degrees, and about 270 degrees relative to the central axis 202. In another exemplary aspect, the at least one capture assembly 250 can comprise three capture assemblies. In this aspect, it is contemplated that in the deployed position, the three capture assemblies can be spaced apart by about 8 cm relative to adjacent capture assemblies, thereby covering 24 cm, the approximate full length of an adult esophagus. Although sample capture devices having three capture assemblies are specifically described herein, it is contemplated that the at least one capture assembly 50 can comprise any number of capture assemblies, such as, for example and without limitation, 2, 4, 5, 6, 7, 8, 9, 10, 11, 12, 13, 14, 15, 16, 17, 18, 19, 20, 21, 22, 23, or 24 capture assemblies. It is further contemplated that the capture assemblies 250 can be substantially equally axially spaced relative to the central axis 202. In still further exemplary aspects, the plurality of buckling elements 252 can comprise three buckling elements, and the at least one capture assembly 250 can comprise three capture assemblies as disclosed herein, thereby forming a 3×3 array of buckling elements and/or capture elements 260. In still further exemplary aspects, the plurality of buckling elements 252 can comprise four buckling elements, and the at least one capture assembly 250 can comprise three capture assemblies as disclosed herein, thereby forming a 3×4 array of buckling elements and/or capture elements 260. FIGS. 22 and 23 display exemplary comparison maps of esophageal disease obtained using 3×3 and 3×4 arrays of buckling elements 252, respectively. FIGS. 22A and 23A display an initial disease map, whereas FIGS. 22B and 23B display a later disease map (following disease progression and/or treatment). The three capture assemblies are respectively labeled as “proximal,” “mid,” and “distal” capture assemblies.

Optionally, the plurality of buckling elements 252 of each capture assembly 250 can be positioned in a substantially staggered configuration, as disclosed herein with respect to capture device 10. For example, it is contemplated that at least one buckling element 252 of the plurality of buckling elements can be offset from another buckling element of the plurality of buckling elements relative to the central axis 202.

In exemplary aspects, it is contemplated that the capture elements 260 can be larger than the buckling elements 252 to which they are secured, as disclosed herein with respect to sample capture device 10. In these aspects, it is contemplated that each capture element 260 can be secured to a buckling element 252 at a first point proximate a crimping element and/or the elongate conduit 214 and at a second point that protrudes outwardly relative to the first point when the capture assembly is in the deployed position. Alternatively, it is contemplated that each capture element 260 can be secured to a buckling element 252 at a first point and to a portion of the elongate conduit 214 at a second point. As disclosed with respect to sample capture device 10, it is contemplated that, in these configurations, when the buckling elements buckle (and the capture assembly is in the deployed position), the capture elements can be configured to angle outwardly, thereby maximizing tissue collection. It is further contemplated that the capture elements 260 can be angularly oriented relative to adjacent capture elements as measured relative to a plane transverse to the central axis 12.

In exemplary aspects, and with reference to FIG. 10, the sample capture device 200 can further comprise first and second actuators 300, 302 configured for engagement by at least a portion of the hand of a user. In these aspects, the first actuator 300 can comprise at least one engagement portion 301, and the second actuator 302 can comprise at least one engagement portion 303. Optionally, each engagement portion 301, 303 can define at least one opening configured to receive one or more fingers of a user. It is contemplated that the first actuator 300 can be operatively coupled to the first central wire such that movement of the actuator 300 relative to the central axis 202 effects a corresponding axial movement of the first central wire 230, and the second actuator 302 can be operatively coupled to the second central wire 232 such that movement of the actuator relative to the central axis effects a corresponding axial movement of the second central wire.

As shown in FIG. 10A, in exemplary configurations, when the first and second actuators 300, 302 are positioned at an initial, neutral position, the at least one sheath 340 can be positioned in the closed position (with the at least one capture assembly 350 being positioned within a respective sheath). As shown in FIG. 10B, in exemplary configurations, when the first actuator is pushed down or depressed (thereby axially advancing the at least one sleeve 340), the at least one sheath 340 can be positioned in the open position, thereby exposing the at least one capture assembly 350. As shown in FIG. 10C, in exemplary configurations, with the first actuator depressed, the second actuator can be pulled up or retracted (thereby pulling the capture assembly 350 upwardly) to position the at least one capture assembly 350 in the deployed position.

As shown in FIG. 10, it is contemplated that the actuators 300, 302 can be provided as part of an assembly. In one aspect, the assembly can comprise a barrel 304 positioned in operative communication with the first end 220 of the elongate conduit 214 and configured to receive at least a portion of the actuators 300, 302 such that the actuators can be selectively axially advanced within both the barrel and the elongate conduit. Optionally, in exemplary aspects, the assembly can further comprise one or more secondary engagement portions 305 configured for engagement with one or more fingers of a user. For example, in these aspects, it is contemplated that the secondary engagement portions 305 can define respective openings configured to receive one or more fingers of a user. In additional aspects, the assembly can optionally comprise a coupler 306 configured to provide a secure, fluid-tight connection between the barrel 304 and the first end 220 of the elongate conduit 214.

Optionally, in one aspect, the sample capture device 200 can further comprise a pH probe as disclosed herein with respect to sample capture device 10. In this aspect, it is contemplated that the sample capture device 200 can optionally have a microchip and/or light as disclosed herein with respect to sample capture device 10.

In another optional aspect, the sample capture device 200 can further comprise a camera as disclosed herein with respect to sample capture device 10.

Optionally, in another aspect, the sample capture device 200 can further comprise a terminal bead as disclosed herein with respect to sample capture device 10.

In still another optional aspect, it is contemplated that each buckling element 252 of the plurality of buckling elements can comprise at least one shim element as disclosed herein with respect to sample capture device 10.

Optionally, in additional exemplary aspects, each buckling element 252 of the plurality of buckling elements of each capture assembly 250 can comprise at least one enclosing element as disclosed herein with respect to sample capture device 10. In exemplary aspects, it is contemplated that each buckling element 252 of the plurality of buckling elements of each capture assembly 250 can comprise at least one shim element and at least one enclosing element as disclosed herein with respect to sample capture device 10.

In exemplary aspects, and with reference to FIGS. 20C-20D, it is contemplated that the capture elements 260 can be directly secured to the second central wire 232, and the plurality of buckling elements 252 can be directly secured to the first central wire 230. In these aspects, it is contemplated that a first end of a respective capture element 260 can be secured to a first crimping element 270 (which is also secured to the second central wire 232), that a second end of the capture element 260 can be secured to a first end of a respective buckling element 252, and that the second end of the buckling element can be secured to a second crimping element 272 (which is also secured to the first central wire 230), as shown in FIGS. 20C-20D. It is contemplated that the capture elements 260 and buckling elements 252 can be secured to one another and to the crimping elements 270, 272 such that the capture elements and buckling elements are both angled outwardly relative to the central axis 202. As shown in FIG. 20C, it is contemplated that in the retracted position, the respective capture assemblies can overlap with adjacent capture assemblies. As shown in FIG. 20D, upon retraction of the second central wire 232, the crimping elements are moved closer together, and the capture elements and buckling elements extend farther outwardly relative to the central axis 202. It is contemplated that these exemplary arrangements can function as a series of flexible joints.

Unless otherwise indicated, it is contemplated that the components and materials disclosed herein with reference to capture device 10 can likewise be used in conjunction with capture device 200.

In exemplary aspects, it is contemplated that one or more of the components of the disclosed sample capture device 200 can be provided in the form of a kit.

### Introduction of the Mucosal Tissue Sample Capture Device Into a Subject

In exemplary aspects, the mucosal tissue sample capture device 10, 200 can be inserted into the esophagus of the subject to permit recovery of one or more mucosal tissue samples. Initially, an entry area (e.g., the nasal passages or the back of the throat of the subject) can be anesthetized using conventional methods. For example, a tincture of lidocane can be applied to the nasal passages and/or back of the throat of the subject. Optionally, at least a portion of the capture device can be coated with a lubricant as is known in the art. The capture device can then be inserted through the nose into the back of the throat with the capture assemblies in the retracted position. Once the capture device reaches a preferred depth, the capture elements can be deployed and/or extended until the capture elements reach the deployed position. The user can selectively pull up and/or twist the capture device to thereby maximize collection of esophageal tissue samples. In exemplary aspects, when the capture device comprises a single capture assembly at the insertional end of the elongate conduit (see FIGS. 3-4, for example), the user can twist the capture device to maximize sample collection. In exemplary aspects, when the capture device comprises a plurality of capture assemblies, the user can pull the device straight up to maximize sample collection. In these aspects, it is contemplated that the capture device can be continually pulled upwardly or, alternatively, the capture device can be pulled up and pushed back down in a user-selected pattern to maximize capture of the cellular debris on the mucosal surface. In either case, the capture elements can be returned to the retracted position before the capture device crosses the upper esophageal sphincter and is removed from the esophagus.

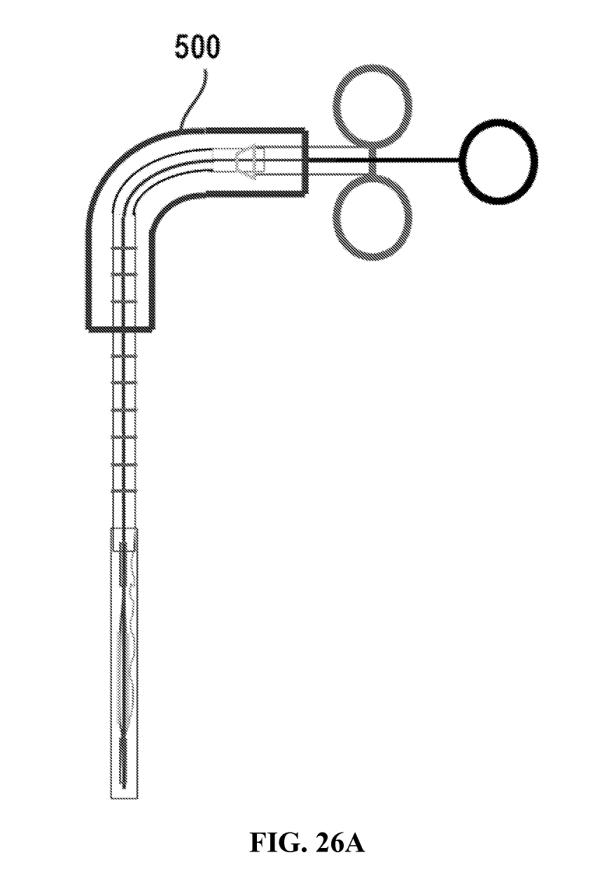

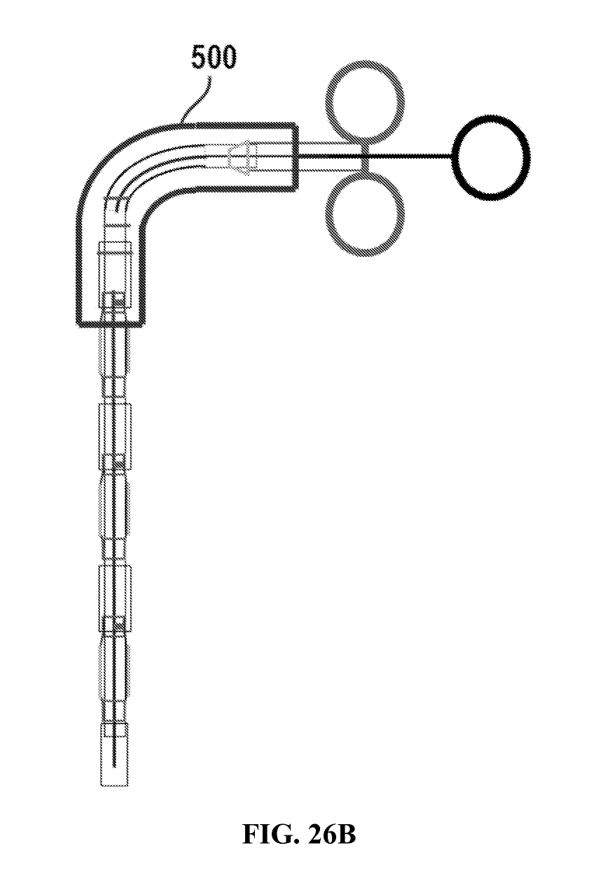

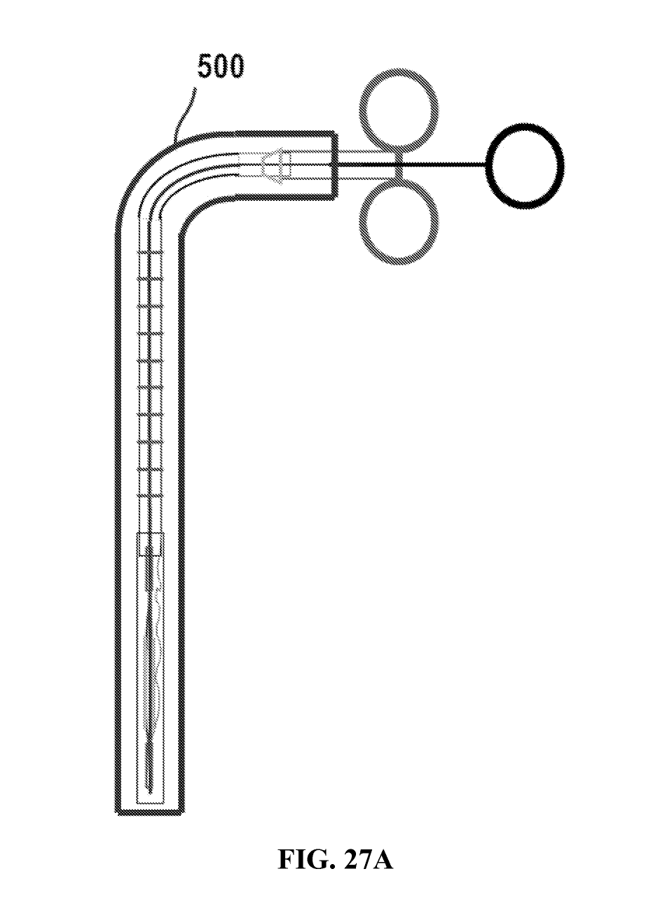

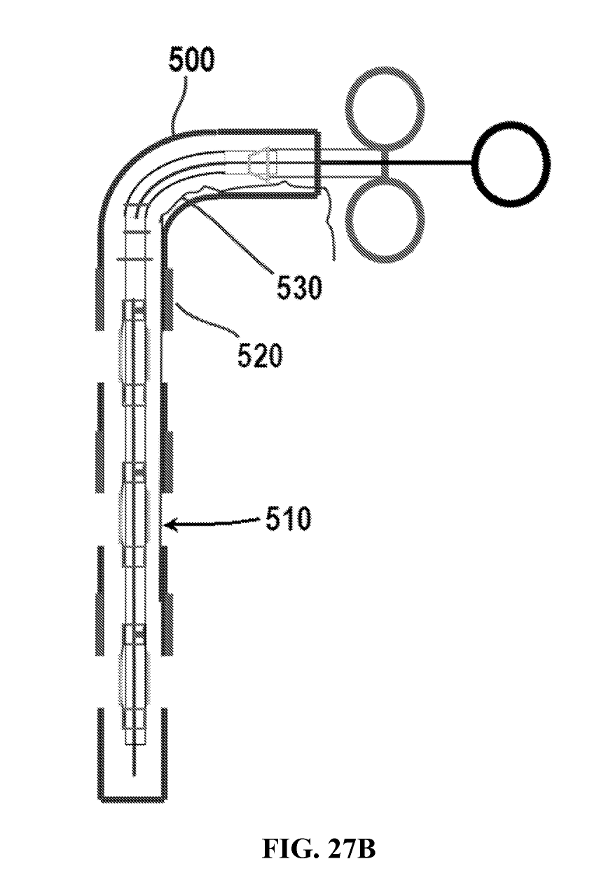

In exemplary aspects, as shown in FIGS. 26 and 27, the capture device 10, 200 can be introduced into the esophagus of the subject using an unconnected outer sheath 500. In these aspects, after anesthetization, the outer sheath 500 can optionally be lubricated. The unconnected outer sheath 500 is then inserted through the nose into the back of the throat. The sheath 500 can optionally be inserted (a) only through the upper esophageal sphincter or (b) down the full depth of the esophagus. For full-depth insertion of the sheath 500, a depth probing device (as is known in the art) or prior knowledge of the approximate depth (e.g. from endoscopy) are required. After the depth probing device is inserted, the depth of the lower esophageal sphincter or surrogate (e.g., bottom of stomach) is registered, and then the depth probing device is removed leaving the outer sheath 500. The capture device can then be inserted, and the capture assemblies can be deployed and retracted as described above.

It is contemplated that the outer sheath 500 can be configured to protect the capture device from unnecessary contamination. It is further contemplated that the outer sheath 500 can be configured to permit use of the capture device with other probes. In exemplary aspects, as shown in FIGS. 26A-26B, the outer sheath 500 can only extend along a portion of the length of the capture device. In other exemplary aspects, as shown in FIG. 27A, the outer sheath 500 can extend along substantially the entire length of the capture device. Optionally, in these aspects, the outer sheath 500 can define a plurality of portals 510 that are spaced along the length of the outer sheath (and the capture device). It is contemplated that the portals can be positioned in alignment with the capture assemblies such that when the capture assemblies are positioned in the deployed position, the capture assemblies extend outside the outer sheath 500. Optionally, the outer sheath 500 can comprise a plurality of covering portions 520 that are selectively between a closed position and an opened position. In the closed position, it is contemplated that the covering portions can substantially close off or cover the portals 510. In the open position, it is contemplated that the covering portions can be axially displaced relative to the central axis of the capture device such that the portals 510 are exposed. In exemplary aspects, the plurality of covering portions 520 can be operatively coupled to a wire 530 that is axially moveable relative to the central axis of the capture device such that the covering portions can be selectively moved between the closed and opened positions. The positioning of the covering portions 520 in the opened position is depicted in FIG. 27B.

In various methods, the sheath and depth probing device can enter together, with the sheath stopping near the upper esophageal sphincter as the depth probing device traverses to or through the lower esophageal sphincter. The depth probing device can be removed through the outer sheath, and the capture device can be inserted through the same sheath. After the tissue sample is captured, the capture device can be removed together with the sheath or both can be removed in a sequential fashion. In various methods, the capture device can be provided with an integrated depth probing element as is known in the art. In these aspects, the sheath and the capture device can be inserted together. The outer sheath can traverse to near the upper esophageal sphincter. The capture device can traverse to near the lower esophageal sphincter and can be removed together or sequentially with the sheath.

### Methods of Using the Mucosal Tissue Sample Capture Device

Also provided is a method of diagnosing eosinophilic esophagitis in a subject, comprising detecting an eosinophil granule protein in the mucosal tissue of the esophagus in a subject, comprising: a) obtaining a mucosal tissue sample from the esophagus in the subject using the disclosed sample capture device; b) contacting the mucosal tissue sample with a detectable composition ex vivo under conditions wherein the detectable composition can bind to an eosinophil granule protein to form a detectable composition/eosinophil granule protein complex; and c) detecting the detectable composition/eosinophil granule protein complex in the mucosal tissue sample of the esophagus, whereby detecting the detectable composition/eosinophil granule protein complex in the mucosal tissue sample of the esophagus diagnoses eosinophilic esophagitis in the subject.

As used herein, a “mucosal tissue” is a tissue lining various cavities within the body. Examples of a mucosal tissue include, but are not limited to, mucosal tissue lining the nose, sinuses, bronchi, lungs, conjunctiva, oral cavity, tongue, esophagus, stomach, pylorus, duodenum, jejunum, ileum, ascending colon, caecum, appendix, transverse colon, descending colon, rectum, anus, urethra, and urinary bladder. A mucosal tissue comprises a mucus layer, an epithelial surface comprising epithelial cells, glandular epithelial cells which secrete mucus, basement membrane, and submucosa with connective tissue. Further, a mucosal tissue sample can comprise one or more of individual or clumped cells, cell debris, granules, and various proteins or molecules found in a cell. A detectable composition/eosinophil granule protein complex can be detected ex vivo in the mucus layer, on the epithelial surface, in the glandular epithelium, on or in the basement membrane, or in the submucosal connective tissue of a mucosal tissue in a subject. In one aspect, a mucosal tissue is from the esophagus of a subject.

As used herein, a “detectable composition” is a composition that can be detected using methods well known in the art. For example, in one aspect, a detectable composition can be a composition tagged with a radiolabel that can be detected by an instrument or device capable of detecting radiation. An example of such a detectable composition is a radiolabeled antibody that can bind to a protein to allow detection of the protein. In another aspect, a detectable composition can be a composition that fluoresces when stimulated by a particular wave-length of light. An example of such a fluorescent composition is fluorescein sodium, which can fluoresce when exposed to ultraviolet light (i.e., black light). In another aspect, a detectable composition can be a solution that can change color when contacted with a particular substance. An example of a detectable composition is a gold nanoparticles colloidal solution, which when contacted with major basic protein 1 changes color. Another example of a solution that can be a detectable composition is a solution that can change color and/or intensity when contacted with eosinophil peroxidase. Examples of detectable compositions are disclosed in the Examples below.

As used herein, an “eosinophil granule protein” is a protein that comprises the granules in eosinophils. When an eosinophil is activated, granule proteins are released from the cell into the surrounding tissue and expressed on the cell surface. The released granule proteins can cause pathologic allergenic inflammatory responses in the surrounding tissue, for example esophageal mucosal tissue. Examples of eosinophil granule proteins include, but are not limited to, major basic protein (MBP), major basic protein 1 (MBP-1), major basic protein 2 (MBP-2), eosinophil derived neurotoxin (EDN), eosinophil cationic protein (ECP), and eosinophil peroxidase (EPO). Other examples of eosinophil granule proteins and targetable eosinophil proteins are provided in Kita et al., Biology of Eosinophils, Chapter 19 of Immunology, which is hereby incorporated by reference for its teaching of examples of eosinophil granule proteins.

In one aspect, an eosinophil granule protein can be EPO. In another aspect, an eosinophil granule protein can be MBP-1.

A detectable composition can contact an esophageal mucosal tissue sample and bind to an eosinophil granule protein in the esophageal mucosal tissue sample ex vivo to form a detectable composition/eosinophil granule protein complex. In one aspect, a detectable composition can be an EPO reagent. For example, an EPO reagent can contact an esophageal mucosal tissue sample, obtained by the disclosed sample capture device, ex vivo. When an esophageal mucosal tissue sample comprising EPO contacts an EPO reagent, the EPO reagent binds to or reacts with EPO and then changes color and/or intensity to orange, leading to a higher absorbance measurement at 492 nm. Thus, a person of skill can quickly diagnose EoE in a subject after detecting a color change in the EPO reagent. Examples of EPO reagents and methods that can be used to detect EPO in a mucosal tissue sample are disclosed below in Example 1.

The disclosed sample capture device has a plurality of capture elements that can be identified with regard to where along the length of the esophagus (i.e., proximal segment, middle segment, or distal segment) in a subject the capture elements obtained one or more mucosal tissue samples. Thus, a person of skill can not only detect ex vivo the presence of a detectable composition/eosinophil granule protein complex, for example EPO reagent/EPO complex, but also can determine the location of the inflammation in the esophagus in a subject by determining from which segment of the esophagus the inflamed tissue sample was obtained.

In another aspect, fluorescein sodium can contact an esophageal mucosal tissue sample, obtained by the disclosed sample capture device, ex vivo to form a fluorescein sodium/MBP-1 complex. In an aspect, one or more capture elements of the disclosed sample capture device can be contacted ex vivo with fluorescein sodium and then rinsed to remove unbound fluorescein sodium. An ultraviolet light (i.e., black light) can then be directed onto the esophageal mucosal tissue sample adherent to the one or more capture elements to detect fluorescence of the fluorescein sodium/MBP-1 complex. When the fluorescein sodium/eosinophil granule protein complex can be detected in the esophageal mucosal tissue sample, the diagnosis of EoE can be made. Examples of fluorescein sodium solutions and methods that can be used to detect MBP-1 in a mucosal tissue sample are disclosed below in Example 2.

In another aspect, a detectable composition can be a gold nanoparticle colloidal solution. For example, a gold nanoparticle colloidal solution can contact an esophageal mucosal tissue sample, obtained by the disclosed sample capture device, ex vivo. When an esophageal mucosal tissue sample comprising, for example, MBP-1 contacts a gold nanoparticle colloidal solution, the gold nanoparticle colloidal solution binds to MBP-1 and changes color. Thus, a person of skill can quickly diagnose EoE in a subject after detecting a color change in the gold nanoparticle colloidal solution. Examples of gold nanoparticle colloidal solutions and methods that can be used to detect MBP-1 in a mucosal tissue sample are disclosed below in Example 2.

Further disclosed is a method of detecting a change in EoE in a subject diagnosed with EoE, comprising: (a) producing a first map of the esophagus in a subject diagnosed with EoE according to the disclosed methods, (b) producing a second map of the esophagus in the subject of step (a) according to the disclosed methods, and (c) comparing the map of step (b) with the map of step (a), whereby detecting a change in the map of step (b) compared to the map of step (a) detects a change in EoE in the subject.

Subsequent esophageal mucosal tissue sampling using the disclosed sample capture device and ex vivo testing according to the disclosed methods can be used to monitor the course of EoE in a subject before, during, and after treatment. Thus, if after initiation of treatment for EoE a subsequent map of a subject's esophagus shows fewer areas of inflammation, a person of skill can determine that the treatment is effective. Conversely, if after initiation of treatment for EoE a subsequent map of a subject's esophagus shows no change or more areas of inflammation, a person of skill can determine that the treatment is not effective.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight; temperature is in ° C. or is at ambient temperature; and pressure is at or near atmospheric.

### Example 1

### EPO Assay

The EPO assay can detect the eosinophil granule protein, eosinophil peroxidase (EPO). The colorimetric EPO assay can be used with modifications. This assay is quantitative and sensitive.

One of the modifications to the colorimetric EPO assay is that it must be substantially accelerated to be clinically relevant. Acceleration strategies can be used including but not limited to temperature, combinations, and changes in the reagent concentrations.

Another modification to the colorimetric EPO assay is the inclusion of NaCN or KCN to block at least myeloperoxidase, a potentially confounding agent.

The use of sponges, foams, microfibers, synthetic fibers, expandable foam, swelling sponges with or without coatings such as polyanions and heparin as contacting elements to reduce background, enhance capture, and make the assay more quantitative and accurate is another modification.

### Materials and Methods

The detection solution, or EPO reagent, is prepared by adding 800 μL of 5 mM o-phenylenediamine (OPD) in 1 M Tris buffer (pH 8.0), 100 μL of sodium cyanide (NaCN) and 1.25 μL 30% H₂O₂.

The patient sample is added to the detection solution (EPO reagent). The reaction is stopped by adding 100 μL of 4 M sulfuric acid and the absorbance of the solution is measured at 492 nm. In the presence of EPO granule protein, the detection solution changes color to orange, leading to higher absorbance measurement at 492 nm.

### Development of EPO Assay

Different concentrations of EPO in 1× PBS (stock solution of 5.3 mg/mL) were prepared. Different capture element material samples were incubated with EPO for 2 minutes and then dipped in the detection solution. Results from different dilutions of EPO stock solution in 1× PBS were examined including the following dilutions 1:10, 1:100, 1:1000, 1:10,000, 1:100,000 and a control sample containing just 1× PBS. The 1:10 dilution sample had the darkest color or intensity; the color or intensity lightened as the dilution increased. Thus, the 1:10 dilution sample was the darkest, and the control sample was the lightest (or most clear).

Frozen brush, or capture element, samples from patients can be used. In one experiment, sample capture device samples from a normal patient having an all eosinophil count of 0, sample capture device samples from an EoE patient having eosinophil counts of Proximal=67, Mid=39, and Distal=73, and control samples (PBS) were used. The samples were incubated with EPO reagent for 30 minutes, and the reaction was stopped by adding sulfuric acid. The sample from the normal patient was darker in color than the control but was lighter in color compared to the sample from the EoE patient. It only took a couple of minutes for the color to change in the EoE patient sample.

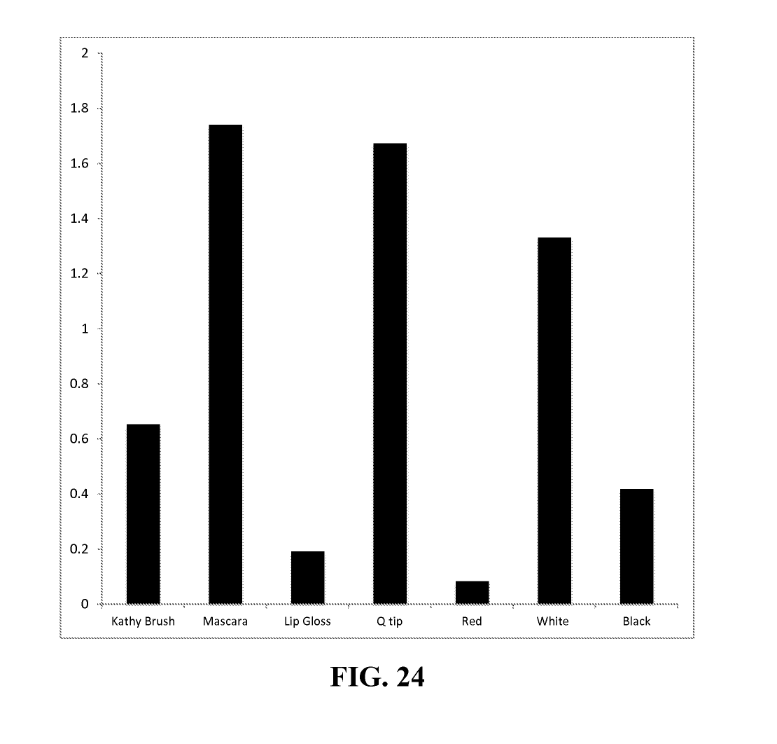

Different sample capture device materials can be used: The lower the background noise (color change from the capture element itself) is, the better the EPO detection system is. FIG. 24 shows the results of the different capture element materials on absorbance or color change.

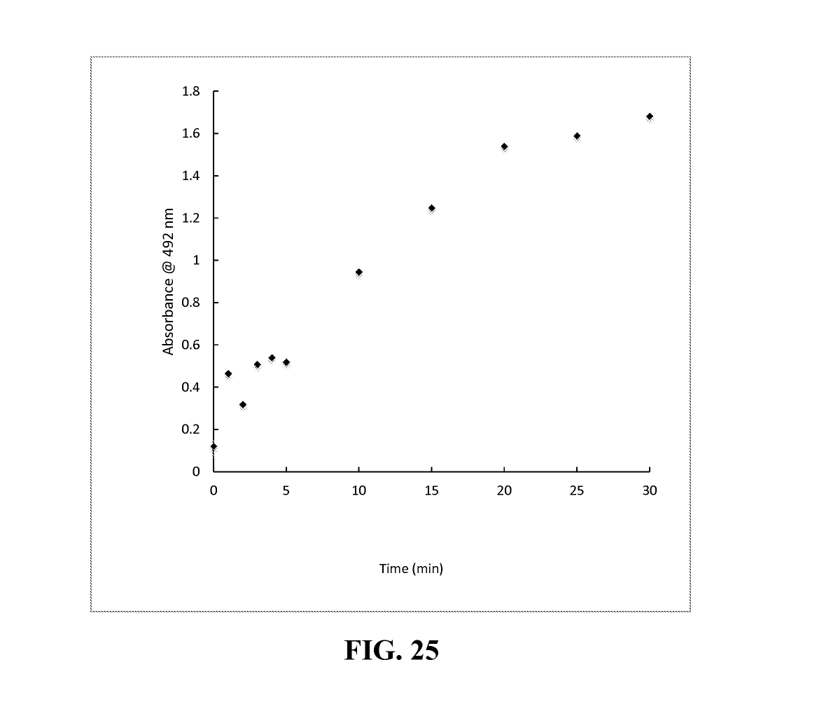

FIG. 25 shows that over time, a 1:1000 dilution of EPO stock solution in 1× PBS gets darker but that it changes color within the first few minutes.

### Example 2

### MBP-1 Assays

The sample capture device can also be used in assays to detect the eosinophil granule protein, major basic protein 1 (MBP-1).

### Gold Nanoparticle Assay

The gold nanoparticle assay can be used to detect MBP-1. The gold nanoparticle colloidal solution changes color in the presence of MBP-1.

### i. Materials and Methods

The detection solution is a gold nanoparticle colloidal solution of size between 10 nm and 60 nm. The patient sample is added to the detection solution (Gold nanoparticle solution). The solution changes color in the presence of MBP-1.

### ii. Development of the Gold Nanoparticle Assay

MBP-1 stock solution of 2.7 mg/mL was used to make 1:10, 1:100, 1:1000, 1:10,000 and 1:100,000 dilution samples. The capture elements were incubated first in the MBP-1 solution for a couple of minutes and then dipped into the detectable composition, i.e., the gold nanoparticle colloidal solution. No change is detected lower than 1:100 dilution of MBP-1

### Fluorescein Assay

The fluorescein assay can be used to detect MBP-1. Fluorescein binds to MBP-1, eosinophil's most dominant granule protein. The higher the intensity of the fluorescein sodium is, the higher the MBP-1 concentration is.

### i. Materials and Methods

The detection solution is a Fluorescein sodium salt solution. The patient sample is dipped into the detectable composition (i.e., Fluorescein solution) and then washed in a 1× PBS solution. The capture elements change color to green under ultraviolet light (black light) in the presence of MBP-1.

### ii. Development of Fluorescein Assay

MBP-1 stock solution of 2.7 mg/mL was used. The capture elements were dipped quickly in the fluorescein detection solution and immediately washed in the 1× PBS. The intensity of the capture elements was checked under the black light.

Different MBP-1 and fluorescein concentrations were examined under black light. The higher the MBP-1 concentration is, the higher the intensity of the fluorescein is after a washing step with 1× PBS.

It was also determined that 1:10, 1:100, 1:1,000, 1:10,000, 1:100,000 dilutions of MBP-1 stock solution and PBS only had different amounts of fluorescence under black light. The fluorescence decreased as the dilution increased. Thus, the 1:10 dilution sample showed the most fluorescence, and the PBS only sample had similar fluorescence or less fluorescence than the 1:10,000 and 1:100,000 dilutions.

### Example 3

### Chemometric Assay

This assay can be designed to distinguish EoE and its phenotypes, resolved EoE, and normal (never diseased) patients. The full spectrum of UV and fluorescence spectroscopy can be used to determine which portion(s) of the spectra most directly distinguish these cases in binary decision sets. For example, the first question distinguishes normal from EoE in any form (or normal/resolved from active disease). The second question can distinguish active EoE from resolved EoE. The third question can distinguish the phenotypes.

This assay can also be used to distinguish the full range of esophageal diseases including but not limited to EoE, Barrett's esophagus, esophageal cancer, GERD, and esophageal candida (i.e., yeast infection). Additional experimentation/assays and brushes can be performed for each of these diseases.

A patient's sample can be incubated (dipped) into the lx PBS media (or any other relevant media or buffer). After the material is removed from the bristles or the capture elements of the sample capture device, the solution is mixed and centrifuged. The clear solution is put into the UV-Vis system to get the full spectrum absorbance. The UV-Vis can be blanked with 1× PBS media as a reference solution. Based on the absorbance range, the disease state can be concluded.

Using a cytology brush as the sample capture device, samples from six patients were assayed using the chemometric assay.

### REFERENCES

Abu-Ghazaleh R I, et al., “Eosinophil granule proteins in peripheral blood granulocytes,” J Leukoc Biol 1992, 52: 611-618.

Attwood S E, et al., “Esophageal eosinophilia with dysphagia. A distinct clinicopathologic syndrome,” Dig Dis Sci 1993, 38(1): 109-116.

Esquerre J P, et al., “Kinetics of technetium-labeled heparin in thromboembolism: Preliminary Report,” Int. J Nucl. Med. Biol., 1979: 6 (4):215-220.

Fogg M I, et al., “Pollen and eosinophilic esophagitis,” J Allergy Cin Immunol 2003, 112(4): 796-797.

Frigas E, et al., “Cytotoxic effects of the guinea pig eosinophil major basic protein on tracheal epithelium,” Lab Invest 1980, 42: 35-43.

Gangotena F, et al., “Eosinophilic Esophagitis, Ringed Esophagus: The Diagnostic Conundrum,” Am J Gastroenterol 2007, 102: S145-S146, Abstract 79.

Gleich G J et al., “Biochemical and functional similarities between human eosinophil-derived neurotoxin and eosinophil cationic protein: homology with ribonuclease,” Proc Natl Acad Sci USA May, 1986, 83: 3146-3150.

Gleich G J et al., “Comparative properties of the Charcot-Leyden crystal protein and the major basic protein from human eosinophils,” J Clin Invest March, 1976, 57: 633-640.

Gleich, G J et al., “Physiochemical and biological properties of the major basic protein from guinea pig eosinophil granules,” J Exp Med 1974, 140: 313-332.

Gonsalves N et al., “A Prospective Trial of Six Food Elimination Diet and Reintroduction of Causative Agents in Adults with Eosinophilic Esophagitis,” Digestive Disease Week Presentation 2008, Abstract No. 727.

Gonsalves N et al., “Histopathologic variability and Endoscopic Correlates in Adults with Eosinophilic Esophagitis,” Gastrointestinal Endosc 2006, 64(3): 313-319.

Gonsalves N et al., “Prospective Clinical Trial of Six Food Elimination Diet or Elemental Diet in the Treatment of Adults with Eosinophilic Gastroenteritis,” Digestive Disease Week Presentation 2009, Abstract No. S1861.

Gonsalves N, Kahrilas P, “Eosinophilic Esophagitis in Adults,” Am J Gastroenterol Clin N Am 2008, 37: 349-368.

Gundel R H et al., “Human eosinophil major basic protein induces airway constriction and airway hyperresponsiveness in primates,” J Clin Invest, 1991, 87: 1470-1473.

Hiebert L M, et al., “Tissue distribution and antithrombotic activity of unlabeled or C14-labeled porcine intestinal mucosal heparin following administration to rats by the oral route,” Can J Physiol Pharmacol., 2000;78: 307-320.

Hirash J, et al., “Heparin and low-molecular-weight heparin: mechanisms of action, pharmacokinetics, dosing, monitoring, efficacy, and safety,” Chest. 2001, 119: 64S-94S.

Hsu, “Principles of heparin-coating techniques,” Perfusion 1991, 6: 209-219.

ICRP, 2008. Radiation Dose to Patients from Radiopharmaceuticals—Addendum 3 to ICRP Publication 53. ICRP Publication 106. Ann. ICRP 38 (1-2).

Kagawalla A F et al., “Effect of 6 food Elimination diet on clinical and histologic outcomes in eosinophilic esophagitis,” Clin Gastroenterol Hepatol 2006, 4: 1097-1102.

Kato M et al., “Eosinophil infiltration and degranulation in normal human tissue,” Anat Record 1998, 252: 418-425.

Kephart G M et al., “Marked deposition of eosinophil-derived neurotoxin in adult patients with eosinophilic esophagitis,” Am J Gastroenterol 2010, 105(2): 298-307.

Konikoff et al., “A Randomized, Double-blind, Placebo controlled trial of Fluticasone Propionate for Pediatric Eosinophilic Esophagitis,” Gastroenterol 2006, 131: 1381-1391.

Kulkami P V, et al., “Technetium Labeled Heparin: Preliminary Report of a New Radiopharmaceutical with Potential for Imaging Damaged Coronary Arteries and Myocardium,” J Nucl Med 1978, 19: 810-815.

Kulkami P V, et al., “Modified technetium-99m heparin for the imaging of acute experimental myocardial infarcts,” J Nucl Med. 1980, 21: 117-121.

Laforest M D, et al., “Pharmacokinetics and biodistribution of technetium 99m labelled standard heparin and a low molecular weight heparin (enoxaparin) after intravenous injection in normal volunteers,” Br J Haematol. 1991, 77: 201-208.

Levine et al., “Disease of the Esophagus: Diagnosis with Esophagography,” Radiology 2005, 237: 414-427.

Liacouras C A et al., “Eosinophilic Esophagitis: a 10-year experience in 381 children,” Clin Gastroenterol hepatol 2005, 3: 1198-1206.

Liacouras C, et al., “Eosinophilic Esophagitis: Updated Consensus Recommendations for children and adults,” J Allergy Clin Immunol. 2011, 128(1): 3-20.

Mackenzie S et al., “Eosinophilic Oesophagitis in Patients Presenting with Dysphagia—A Prospective Analysis,” Aliment Pharmacol Therapeutics 2008, 28(9): 1140-1146.

Majdalani G, et al., “Kinetics of technetium-labeled heparin in hemodialyzed patients,” Kidney Int. Supply., 1993:41:S131-134.

Markowitz J et al., “Elemental Diet is Effective Treatment for Eosinophilic Esophagitis in Children and Adolescents,” Am J Gastroenterol 2003, 98: 777-782.

Mishra A et al., “An etiological role for aeroallergens and eosinophils in experimental eosinophilic esophagitis,” J Clin Invest 2001, 107: 83-90.

O'Donnell M C et al., “Activation of basophil and mast cell histamine release by eosinophil granule major basic protein,” J Exp Med, 1983, 157: 1981-1991.

Odze, R. D., “Pathology of eosinophilic esophagitis: What the clinician needs to know,” Am J Gastroenterol 2009, 104: 485-490.

Pasha, S F, et al., “Patient characteristics, clinical, endoscopic, and histologic findings in adult eosinophilic esophagitis: a case series and systematic review of the medical literature,” Dis Esophagus 2007, 20(4): 311-319.

Pentiuk, S et al., “Dissociation between symptoms and histological severity in pediatric eosinophilic esophagitis,” J Pediatr Gastroenterol Nutr. 2009, 48: 152-160.

Peters M S et al., “Localization of human eosinophil granule major basic protein, eosinophil cationic protein, and eosinophil-derived neurotoxin by immunoelectron microscopy,” Lab Invest 1986, 54: 656-662.

Peterson K A, et al., “Elemental Diet Induces Histologic Response in Adult Eosinophilic Esophagitis,” Am J Gastroenterol 2013, Epub ahead of print.

Prasad, G A et al., “Secular trends in the epidemiology and outcomes of eosinophilic esophagitis in Olmsted County, Minnesota (1976-2007),” Digestive Disease Week, May 2008.

Saffari H, et al., “Patchy eosinophil distributions in an esophagectomy specimen from a patient with eosinophilic esophagitis: Implications for endoscopic biopsy,” J Allergy Clin. Immunol. 2012, 130: 798-800.

Shah, A et al., “Histopathologic variability in Children with Eosinophilic Esophagitis,” Am J Gastroenterol 2009, 104(3): 716-721.

Straumann A, et al., “Natural history of primary eosinophilic esophagitis: a follow-up of 30 adult patients for up to 11.5 years,” Gastroenterology 2003, 125: 1660-1669.

Swaminathan, G J et al., “Eosinophil-granule major basic protein, a C-type lectin, binds heparin,” Biochem 2006, 44: 14152-14158.

Talley N J, Kephart G M, McGovern T W, Carpenter H A, Gleich G J., “Deposition of eosinophil granule major basic protein in eosinophilic gastroenteritis and celiac disease,” Gastroenterology. 1992, 103: 137-145.

Tantibhaedhyangkul, U et al., “Increased Esophageal Regulatory T Cells and Eosinophil Characteristics in Children with Eosinophilic Esophagitis and Gastroesophageal Reflux Disease,” Annals of Clinical & Laboratory Science 2009, 39: 99-107.

Wagner, L et al., “Eosinophils,” Encyclopedia of Life Sciences, John Wiley & Sons 2006.

Wang F Y et al., “Is there a seasonal variation in the incidence or intensity of allergic eosinophilic esophagitis in newly diagnosed children?” J Clin Gastroenterol Hepatol 2007, 41: 451-453.

Wasmoen, T L et al., “Biochemical and amino acid sequence analysis of human eosinophil granule major basic protein,” J Biol Chem 1988, 263: 12559-12563.

It should be appreciated that the actual and relative angles and dimensions depicted in the Figures may be exaggerated for clarity and, consequently, may not be to scale.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A mucosal tissue sample capture device for insertion within the esophagus of a subject, the sample capture device having a central axis, the sample capture device comprising:
an elongate conduit surrounding the central axis, the elongate conduit defining a central bore and having an outer surface, a first end, and an opposed, insertional end;
a central wire configured for selective movement relative to the central axis, at least a portion of the central wire being positioned within the central bore of the elongate conduit;
at least one sheath operatively coupled to the outer surface of the elongate conduit;
at least one capture assembly operatively coupled to the central wire, wherein selective axial movement of the central wire is configured to effect axial and radial movement of each capture assembly of the at least one capture assembly, each capture assembly of the at least one capture assembly being axially moveable about and between an enclosed position and an open position and being radially moveable about and between a retracted position and a deployed position, wherein each capture assembly of the at least one capture assembly comprises:
a plurality of buckling elements, the plurality of buckling elements being azimuthally spaced relative to the central axis, each buckling element having a first end and an opposed second end; and
a first crimping element operatively coupled to the first ends of the plurality of buckling elements and to the central wire; and
a second crimping element operatively coupled to the second ends of the plurality of buckling elements such that the central wire is axially moveable relative to the second crimping element,
wherein, in the enclosed position, each capture assembly of the at least one capture assembly is positioned within a respective sheath of the at least one sheath,
wherein, in the open position, each capture assembly of the at least one capture assembly is axially advanced beyond a respective sheath such that the capture assembly is positioned outside the sheath, and
wherein, in the deployed position, the first and second crimping elements compress the plurality of buckling elements of each capture assembly such that the plurality of buckling elements extend outwardly relative to the central axis.

2. The sample capture device of claim 1, wherein each buckling element of the plurality of buckling elements has an outer surface, wherein each buckling element of the plurality of buckling elements comprises at least one capture element secured to the outer surface of the buckling element such that the at least one capture element projects outwardly relative to the central axis, and wherein the at least one capture element is configured to capture a mucosal tissue sample of the subject.

3. The sample capture device of claim 2, wherein each capture element of the at least one capture element comprises a sponge material.

4. The sample capture device of claim 2, wherein the at least one capture element comprises a plurality of bristles.

5. The sample capture device of claim 2, wherein at least a portion of each capture element of the at least one capture element is coated with a material configured to promote adhesion of a mucosal tissue sample of the subject to the capture element.

6. The sample capture device of claim 2, wherein the plurality of buckling elements comprises four buckling elements, and wherein the plurality of buckling elements are substantially equally azimuthally spaced relative to the central axis.

7. The sample capture device of claim 2, wherein the at least one capture assembly comprises three capture assemblies, and wherein the capture assemblies are substantially equally axially spaced relative to the central axis.

8. The sample capture device of claim 2, wherein the at least one sheath comprises a first sheath secured to the insertional end of the elongate conduit, wherein the at least one capture assembly comprises a first capture assembly, wherein, in the enclosed position, the first capture assembly is positioned within the first sheath, and wherein the sample capture device further comprises a restraint wire operatively coupled to and extending between the insertional end of the elongate conduit and the second crimping element of the first capture assembly such that the restraint wire limits the axial movement of the first capture assembly beyond the first sheath.

9. The sample capture device of claim 2, wherein the elongate conduit comprises a plurality outer tubes and at least one inner tube, each inner tube of the at least one inner tube being secured within and extending between sequential outer tubes such that the at least one inner tube and the plurality of outer tubes cooperate to define the central bore of the elongate conduit, the plurality of outer tubes defining the outer surface of the elongate conduit, wherein an inner tube of the at least one inner tube is received within the first and second crimping elements of a respective capture assembly of the at least one capture assembly, and wherein each capture assembly further comprises a restraint wire operatively coupled to and extending between the first crimping element of the capture assembly and the central wire.

10. The sample capture device of claim 9, wherein the plurality of outer tubes have an outer diameter, wherein the capture elements of the plurality of buckling elements of each capture assembly of the at least one capture assembly cooperate to define an operative diameter of the capture assembly, and wherein, when the at least one capture assembly is in a deployed position, the operative diameter of the at least one capture assembly is greater than the outer diameter of the plurality of outer tubes such that the plurality of outer tubes restrict axial movement of the at least one capture assembly.

11. The sample capture device of claim 2, further comprising an actuator configured for engagement by at least a portion of the hand of a user, wherein the actuator is operatively coupled to the central wire such that movement of the actuator relative to the central axis effects a corresponding axial movement of the central wire.

12. The sample capture device of claim 2, further comprising a pH probe positioned proximate the insertional end of the elongate conduit.

13. The sample capture device of claim 2, further comprising a camera positioned proximate the insertional end of the elongate conduit.

14. The sample capture device of claim 2, wherein each buckling element of the plurality of buckling elements has an inner surface, wherein each buckling element of the plurality of buckling elements comprises at least one shim element secured to the inner surface of the buckling element, and wherein each shim element of the at least one shim element is secured in opposition to one or more capture elements of the at least one capture element.

15. The sample capture device of claim 2, wherein each buckling element of the plurality of buckling elements of each capture assembly comprises at least one enclosing element secured to the outer surface of the buckling element, the at least one enclosing element being deformable between a closed position and an open position,
wherein the at least one enclosing element is operatively coupled to the buckling element such that when the capture assembly is in the retracted position the at least one enclosing element is in the closed position and when the capture assembly is in the deployed position the at least one enclosing element is in the open position,
wherein, in the closed position, each enclosing element shields at least a portion of a capture element of the at least one capture element,
wherein, in the open position, each enclosing element is retracted to expose at least a portion of a capture element of the at least one capture element.

16. A mucosal tissue sample capture device for insertion within the esophagus of a subject, the sample capture device having a central axis, the sample capture device comprising:
first and second central wires configured for selective movement relative to the central axis;
at least one sheath operatively coupled to the first central wire, wherein selective movement of the first central wire is configured to effect movement of each sheath of the at least one sheath about and between a closed position and an open position;
at least one capture assembly operatively coupled to the second central wire, wherein selective movement of the second central wire is configured to effect movement of each capture assembly of the at least one capture assembly about and between a retracted position and a deployed position, wherein each capture assembly of the at least one capture assembly comprises:
a plurality of buckling elements, the plurality of buckling elements being azimuthally spaced relative to the central axis, each buckling element having a first end and an opposed second end; and
a first crimping element operatively coupled to the first ends of the plurality of buckling elements such that the first and second central wires are axially moveable relative to the first crimping element; and
a second crimping element operatively coupled to the second ends of the plurality of buckling elements and the second central wire such that the first central wire is axially moveable relative to the second crimping element,
wherein, in the closed position of the at least one sheath, each capture assembly of the at least one capture assembly is positioned within a respective sheath of the at least one sheath,
wherein, in the open position of the at least one sheath, each sheath of the at least one sheath is axially advanced relative to a respective capture assembly such that the capture assembly is positioned outside the sheath, and
wherein, in the deployed position, the second crimping element of each capture assembly compresses the plurality of buckling elements of the capture assembly such that the plurality of buckling elements extend outwardly relative to the central axis.

17. The sample capture device of claim 16, wherein each buckling element of the plurality of buckling elements has an outer surface, wherein each buckling element of the plurality of buckling elements comprises at least one capture element secured to the outer surface of the buckling element such that the at least one capture element projects outwardly relative to the central axis, and wherein the at least one capture element is configured to capture a mucosal tissue sample of the subject.

18. The sample capture device of claim 17, wherein each capture element of the at least one capture element comprises a sponge material.

19. The sample capture device of claim 17, wherein the at least one capture element comprises a plurality of bristles.

20. The sample capture device of claim 17, wherein at least a portion of each capture element of the at least one capture element is coated with a material configured to promote adhesion of a mucosal tissue sample of the subject to the capture element.

21. The sample capture device of claim 17, wherein the plurality of buckling elements comprises four buckling elements, and wherein the buckling elements of the plurality of buckling elements are substantially equally azimuthally spaced relative to the central axis.

22. The sample capture device of claim 17, wherein the at least one capture assembly comprises three capture assemblies, and wherein the capture assemblies are substantially equally axially spaced relative to the central axis.

23. The sample capture device of claim 17, further comprising first and second actuators configured for engagement by at least a portion of the hand of a user, wherein the first actuator is operatively coupled to the first central wire such that movement of the actuator relative to the central axis effects a corresponding axial movement of the first central wire, and wherein the second actuator is operatively coupled to the second central wire such that movement of the actuator relative to the central axis effects a corresponding axial movement of the second central wire.

24. A sample capture device for insertion within the esophagus of a subject, the sample capture device having a central axis, the sample capture device comprising:
at least one capture assembly, each capture assembly of the at least one capture assembly being radially moveable about and between a retracted position and a deployed position, wherein each capture assembly of the at least one capture assembly comprises:
a plurality of buckling elements, each buckling element having a first end and an opposed second end; and
a first crimping element operatively coupled to the first ends of the plurality of buckling elements; and
a second crimping element operatively coupled to the second ends of the plurality of buckling elements; and
means for effecting movement of the at least one capture assembly about and between the retracted position and the deployed position,
wherein, in the deployed position, the first and second crimping elements compress the plurality of buckling elements of each capture assembly such that the plurality of buckling elements extend outwardly relative to the central axis.

25-34. (canceled)
